(19) United States
(12) Patent Application Publication
(10) Pub. No.: US 2025/0391914 A1
(43) Pub. Date: Dec. 25, 2025

(54) THIOCARBONATE COMPOSITIONS FOR LITHIUM-SULFUR BATTERIES

(71) Applicant: Conamix Inc., Ithaca, NY (US)

(72) Inventors: Stephen Burkhardt, Ithaca, NY (US); Jay J. Farmer, Ithaca, NY (US)

(21) Appl. No.: 18/867,414

(22) PCT Filed: May 19, 2023
(86) PCT No.: PCT/US2023/022841

§ 371 Date: Nov. 19, 2024

Related U.S. Application Data
(60) Provisional application No. 63/344,213, filed on May 20, 2022.

(51) Int. Cl. H01M 10/0567; C07C 329/06; C07D 207/46; C07D 209/48; C07D 231/12; C07D 231/16; C07D 233/61; C07D 339/06; C07F 7/18; H01M 10/0525; H01M 10/42

(52) U.S. Cl. CPC H01M 10/0567; C07C 329/06; C07D 207/46; C07D 209/48; C07D 231/12; C07D 231/16; C07D 233/61; C07D 339/06; C07F 7/1804; H01M 10/0525; H01M 10/4235; H01M 2300/0025

(57) ABSTRACT

The present disclosure provides compounds useful as electrolyte materials, and lithium sulfur batteries comprising the same.

### CROSS-REFERENCE TO RELATED APPLICATIONS

This application claims the benefit of U.S. Provisional Application No. 63/344,213, filed on May 20, 2022, which is incorporated herein by reference in its entirety.

### BACKGROUND

There is significant work being conducted to develop lithium ion batteries with high energy density, long cycle life, and low cost, particularly batteries for electric vehicles and consumer electronics.

Sulfur is a low cost, high specific energy material that is a by-product of the oil and gas industry. Sulfur-based battery cathodes have been under investigation for some time. As a high energy density cathode material, sulfur promises to eliminate the need for cobalt and nickel in lithium batteries. Cobalt is expensive, toxic, and its mining in certain regions may be subject to loose regulation and unethical practices. Nickel has high energy density, but there are long term nickel supply concerns, for example, recently motivating Tesla's shift away from nickel-containing battery cells (Lambert, Fred, “Elon Musk says Tesla is shifting more electric cars to LFP batteries over nickel supply concerns,” Feb. 26, 2021, Electrek).

However, manufacture of a practical lithium-sulfur battery has been an elusive goal. Among the numerous challenges that plague sulfur cathodes, one of the most serious arises from the requirements of multi-step conversion of S₈ to Li₂S. While both sulfur and lithium sulfide are highly insoluble, their interconversion proceeds via intermediate lithium polysulfides, Li₂Sₓ which are highly soluble. In a typical sulfur battery containing a liquid electrolyte, formation and interconversion of lithium polysulfides takes place in the solution phase. While aliphatic carbonates are the workhorse liquid electrolytes for current generation lithium ion batteries, they are not stable in lithium sulfur systems. This is a major hindrance for development of sulfur cathodes-most sulfur systems use polyether and cyclic ethers as electrolytes since these do not react appreciably with sulfide nucleophiles, unfortunately, ethers interact with lithium anodes and/or do not form stable SEIs (solid electrolyte interphases), as such there is an acute need for sulfide-stable electrolytes that are also stable with lithium anodes.

### SUMMARY

Presented herein are compositions and compounds for binders, additives for electrolytes, and electrolytes comprising thiocarbonyl functional groups, and batteries including such binders, additives, and electrolytes.

It is a goal of the present disclosure to improve electrochemical cell performance by means of including disclosed binders, additives, and electrolytes in electrochemical cells. For example, without wishing to be bound by any theory, disclosed binders, additives, and electrolytes comprising thiocarbonyl functional groups improves SEI stability when included in electrochemical cells by interacting with polysulfides in the cell and advantageously reducing diffusion rate. Accordingly, the present disclosure provides for, among other things, improved performance characteristics (e.g., coulombic efficiency), of electrochemical cells having disclosed binders, additives, and/or electrolytes comprising thiocarbonyl functional groups.

In one aspect, the present disclosure is directed to a binder for a sulfur cathode comprising a thiocarbonyl functional group.

In some embodiments, the binder comprises thiocarbonyl functional groups of formula X_{y}C═S, where each X is independently selected from oxygen, nitrogen, sulfur, or carbon, and y is 1 or 2; wherein one X can be taken together with the other X and intervening atoms to form a ring; and when y is 1, X is connected to the thiocarbonyl carbon via a double bond. In some embodiments, y is 2. In some embodiments, each X is independently selected from nitrogen, sulfur, or carbon. In some embodiments, each X is independently selected from sulfur or carbon. In some embodiments, y is 1. In some embodiments, wherein y is 1, the binder comprises isothiocyanate functional groups.

In some embodiments, the binder is uncycled.

In another aspect, the present disclosure is directed to an additive for an electrolyte in a lithium sulfur battery comprising a thiocarbonyl functional group.

In some embodiments, the additive comprises thiocarbonyl functional groups of formula X_{y}C═S, where each X is independently selected from oxygen, nitrogen, sulfur, or carbon, and y is 1 or 2; wherein one X can be taken together with the other X and intervening atoms to form a ring; and when y is 1, X is connected to the thiocarbonyl carbon via a double bond. In some embodiments, y is 2. In some embodiments, each X is independently selected from nitrogen, sulfur, or carbon. In some embodiments, each X is independently selected from sulfur or carbon. In some embodiments, y is 1. In some embodiments, wherein y is 1, the additive comprises isothiocyanate functional groups.

In some embodiments, the additive is uncycled.

In another aspect, the present disclosure is directed to an electrolyte in a lithium sulfur battery comprising a thiocarbonyl functional group.

In some embodiments, the electrolyte comprises thiocarbonyl functional groups of formula X_{y}C═S, where each X is independently selected from oxygen, nitrogen, sulfur, or carbon, and y is 1 or 2; wherein one X can be taken together with the other X and intervening atoms to form a ring; and when y is 1, X is connected to the thiocarbonyl carbon via a double bond. In some embodiments, y is 2. In some embodiments, each X is independently selected from nitrogen, sulfur, or carbon. In some embodiments, each X is independently selected from sulfur or carbon. In some embodiments, y is 1. In some embodiments, wherein y is 1, the electrolyte comprises isothiocyanate functional groups.

In some embodiments, the electrolyte is uncycled.

In another aspect, the present disclosure is directed to a compound (e.g., for use in an electrochemical cell, e.g., for use as an additive in an electrolyte for an electrochemical cell, for use as a functional binder, e.g., for use in binder for an electrochemical cell) of Formula I′:

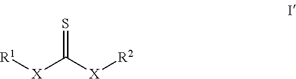

wherein R¹ and R² are each independently hydrogen or an optionally substituted group selected from C₁₋₁₅ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur;
   or R¹ and R² are taken together with intervening atoms to form an optionally substituted ring;
      each X is absent or independently selected from O, S, NR^{z}, and CR³R⁴;
      each R^{z} is independently hydrogen or optionally substituted C₁₋₁₂ aliphatic;
each R³ and R⁴ is independently hydrogen, halogen, —CN, —NO₂, —N(R)₂, —OR, —SR, or an optionally substituted group selected from C₁₋₁₂ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
each R is independently hydrogen, optionally substituted C₁₋₆ aliphatic, optionally substituted 3- to 7-membered saturated or partially unsaturated carbocyclyl, or optionally substituted 3- to 7-membered saturated or partially unsaturated monocyclic heterocyclyl having 1-3 heteroatoms independently selected from nitrogen, oxygen, and sulfur, or two R when attached to the same nitrogen atom are taken together form an optionally substituted 3- to 7-membered saturated or partially unsaturated monocyclic heterocyclyl having 0-2 additional heteroatoms independently selected from nitrogen, oxygen, and sulfur. In some embodiments, R¹ and R² are taken together with intervening atoms to form an optionally substituted ring selected from 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

In some embodiments, R¹ and R² are taken together with intervening atoms to form an optionally substituted 3- to 7-membered saturated or partially unsaturated monocyclic heterocyclyl having 1-3 heteroatoms independently selected from nitrogen, oxygen, and sulfur.

In some embodiments, R¹ and R² are taken together with intervening atoms to form an optionally substituted 5-membered saturated or partially unsaturated monocyclic heterocyclyl having 2 sulfur heteroatoms.

In some embodiments, R¹ and R² are taken together with intervening atoms to form an optionally substituted 5- to 6-membered monocyclic heteroaryl having 1-4 heteroatoms independently selected from nitrogen, oxygen, and sulfur.

In some embodiments, one X is NR^{z}, the other X is independently selected from O, S, NR^{z}, and CR³R⁴, and each of R^{z}, R¹, R², R³, and R⁴ is hydrogen.

In some embodiments, each X is independently NR^{z}, and each of R^{z}, R¹, and R² is independently hydrogen or C₁₋₆ aliphatic.

In some embodiments, each X is independently NR^{z}, and each of R^{z}, R¹, and R² is hydrogen.

In some embodiments, R¹ and R² are taken together with intervening atoms to form Ring A as in Formula II:

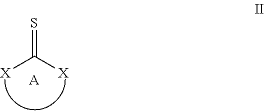

wherein, Ring A is an optionally substituted ring selected from 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

In some embodiments, Ring A is optionally substituted 5-membered saturated or partially unsaturated monocyclic heterocyclyl having 2 sulfur heteroatoms.

In some embodiments, Ring A is optionally substituted 5- to 6-membered monocyclic heteroaryl having 1-4 heteroatoms independently selected from nitrogen, oxygen, and sulfur.

In some embodiments, both X are absent.

In some embodiments, both X are S.

In some embodiments, both X are NR².

In some embodiments, the compound is trithiocyanuric acid.

In some embodiments, a binder, an additive for an electrolyte, or an electrolyte include a compound of any of the embodiments disclosed herein.

In some embodiments, binder, additive, or electrolyte comprising a compound selected from the group consisting of 3H-1,2-benzodithiol-3-one, phenylacetyl disulfide, tetramethylthiourea, thioacetamide, thiourea, trithiocyanuric acid, vinylene trithiocarbonate, zinc dimethyldithiocarbamate, and dimethyl trithiocarbonate.

In some embodiments, a binder, an additive for an electrolyte, or an electrolyte includes a compound of Table 1 disclosed herein.

In some embodiments, an electrolyte composition comprises a compound selected from the group consisting of 3H-1,2-benzodithiol-3-one, phenylacetyl disulfide, tetramethylthiourea, thioacetamide, thiourea, trithiocyanuric acid, vinylene trithiocarbonate, zinc dimethyldithiocarbamate, and dimethyl trithiocarbonate.

In some embodiments, an electrolyte composition includes a compound of Table 1 disclosed herein.

In some embodiments, an electrolyte composition comprises a compound of any of the embodiments disclosed herein.

In some embodiments, the electrolyte composition comprises a compound of Formula III:

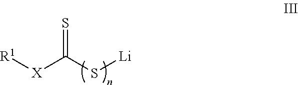

wherein n is 3, 4, 5, 6, 7, or 8.

In some embodiments, the electrolyte composition comprises a compound of Formula IV:

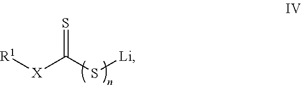

wherein n is 3, 4, 5, 6, 7, or 8.

In some embodiments, the electrolyte composition comprises Li(S)ₙR¹.

In some embodiments, the electrolyte composition comprises Li(S)ₙR².

In some embodiments, X is S.

In some embodiments, a lithium trithiocarbonate is the primary (e.g., the greatest percentage by weight or volume) lithium salt in the electrolyte composition.

In some embodiments, the electrolyte composition is uncycled.

In some embodiments, a lithium sulfur battery comprising a binder, an additive for an electrolyte, or electrolyte of any of the embodiments disclosed herein.

In some embodiments, a lithium sulfur battery comprises a compound of any of the embodiments disclosed herein.

In some embodiments, a lithium sulfur battery comprises an electrolyte composition of any of the embodiments disclosed herein.

In some embodiments, the battery is uncycled.

In another aspect, the present disclosure is directed to a method of making a lithium sulfur battery, comprising adding a binder, an additive for an electrolyte, or an electrolyte of any of the disclosed embodiments, a compound of any of the disclosed embodiments, or an electrolyte composition of any of the disclosed embodiments to a battery encasement, wherein the step is performed prior to charging or discharging.

Any two or more of the features described in this specification, including in this summary section, may be combined to form implementations not specifically explicitly described in this specification.

### Definitions

In order for the present disclosure to be more readily understood, certain terms used herein are defined below. Additional definitions for the following terms and other terms may be set forth throughout the specification.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomic, and geometric (or conformational) mixtures of provided compounds are within the scope of the disclosure. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

About/Approximately: The term “about” or “approximately”, when used herein in reference to a value, refers to a value that is similar, in context, to the referenced value. In general, those skilled in the art, familiar with the context, will appreciate the relevant degree of variance encompassed by “about” or “approximately” in that context. For example, in some embodiments, e.g., as set forth herein, the term “about” can encompass a range of values that within 25% 20%, 19%, 18%, 17, 15%, 14%, 13%, %12%, 11%, 10%, 9%, 8%, 7%, 6%, 5%, 4%, 3%, 2%, 1%, or within a fraction of a percent, of the referred value.

Aliphatic: The term “aliphatic” refers to a straight-chain (i.e., unbranched) or branched, optionally substituted hydrocarbon chain that is completely saturated or that contains one or more units of unsaturation, or a monocyclic or bicyclic hydrocarbon that is completely saturated or that contains one or more units of unsaturation but which is not aromatic (also referred to herein as “carbocyclic” or “cycloaliphatic”), that has a single point of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, alkynyl groups and hybrids thereof. In some embodiments, “aliphatic” refers to a straight-chain (i.e., unbranched) or branched, optionally substituted hydrocarbon chain that is completely saturated or that contains one or more units of unsaturation that has a single point of attachment to the rest of the molecule.

Alkyl: The term “alkyl”, used alone or as part of a larger moiety, refers to a saturated, optionally substituted straight or branched hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Carbocyclyl: The terms “carbocyclyl,” “carbocycle,” and “carbocyclic ring” as used herein, refer to saturated or partially unsaturated cyclic aliphatic monocyclic, bicyclic, or polycyclic ring systems, as described herein, having from 3 to 14 members, wherein the aliphatic ring system is optionally substituted as described herein. Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₇-C₁₀ bicyclic hydrocarbon that is completely saturated or that contains one or more units of unsaturation, but which is not aromatic, that has a single point of attachment to the rest of the molecule. The term “cycloalkyl” refers to an optionally substituted saturated ring system of about 3 to about 10 ring carbon atoms. In some embodiments, cycloalkyl groups have 3-6 carbons. Exemplary monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkenyl: The term “alkenyl”, used alone or as part of a larger moiety, refers to an optionally substituted straight or branched hydrocarbon chain having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Aryl: The term “aryl” refers to monocyclic and bicyclic ring systems having a total of six to fourteen ring members (e.g., C₆₋₁₄), wherein at least one ring in the system is aromatic and wherein each ring in the system contains three to seven ring members. The term “aryl” may be used interchangeably with the term “aryl ring”. In some embodiments, “aryl” refers to an aromatic ring system which includes, but not limited to, phenyl, naphthyl, anthracyl and the like, which may bear one or more substituents. Unless otherwise specified, “aryl” groups are hydrocarbons.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, and “heterocyclic ring” are used interchangeably and refer to a stable 3- to 7-membered monocyclic or 7- to 10-membered bicyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)).

Partially Unsaturated: As used herein, the term “partially unsaturated”, when referring to a ring moiety, means a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Substituted or optionally substituted: As described herein, compounds of this disclosure may contain “optionally substituted” moieties. In general, the term “substituted,” whether preceded by the term “optionally” or not, means that one or more hydrogens of the designated moiety are replaced with a suitable substituent (i.e., as described below for optionally substituted groups). “Substituted” applies to one or more hydrogens that are either explicit or implicit from the structure (e.g.,

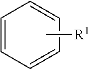

refers to at least

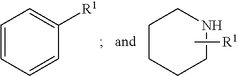

refers to at least

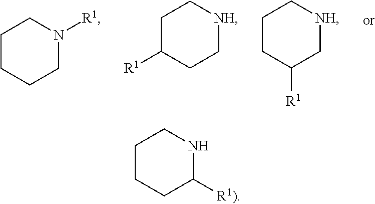

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

### BRIEF DESCRIPTION OF THE DRAWING

Drawings are presented herein for illustration purposes, not for limitation. The foregoing and other objects, aspects, features, and advantages of the disclosure will become more apparent and may be better understood by referring to the following description taken in conjunction with the accompanying drawings, in which:

FIG. 1 is a pictorial representation of a cross section of an electrochemical cell according to certain embodiments of the present disclosure.

FIG. 2 is a pictorial representation of a cylindrical battery according to certain embodiments of the present disclosure.

FIG. 3 is a graphical representation of discharge specific capacity performance characteristics (e.g., at 0.1 c) of a battery according to certain embodiments of the present disclosure.

FIG. 4 is a graphical representation of discharge specific capacity performance characteristics (e.g., at 0.333 c) of a battery according to certain embodiments of the present disclosure.

FIG. 5 is a graphical representation of discharge average voltage performance characteristics of a battery according to certain embodiments of the present disclosure.

FIG. 6 is a graphical representation of pulse average efficiency performance characteristics of a battery according to certain embodiments of the present disclosure.

FIG. 7 is a graphical representation of pulse end of first plateau efficiency performance characteristics of a battery according to certain embodiments of the present disclosure.

FIG. 8 is a graphical representation of pulse end of discharge efficiency performance characteristics of a battery according to certain embodiments of the present disclosure.

FIG. 9 is a graphical representation of coulombic efficiency performance characteristics of a battery according to certain embodiments of the present disclosure.

FIG. 10 is a graphical representation of thermal efficiency performance characteristics of a battery according to certain embodiments of the present disclosure.

FIG. 11 is a graphical representation of voltage efficiency performance characteristics of a battery according to certain embodiments of the present disclosure.

FIG. 12 is a pictorial representation of a coin cell assembly according to one or more embodiments of the disclosure.

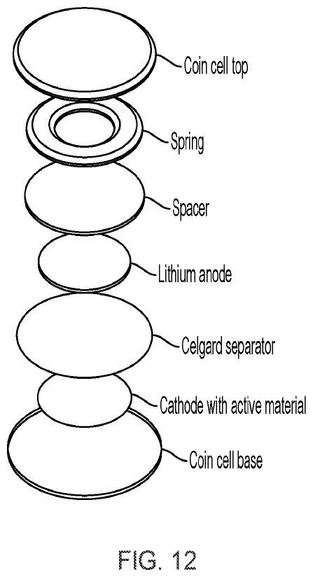

### DESCRIPTION OF CERTAIN EMBODIMENTS

Elements of different implementations described herein may be combined to form other implementations not specifically set forth above. Elements may be left out of the devices described herein without adversely affecting their operation. Various separate elements may be combined into one or more individual elements to perform the functions described herein.

It is contemplated that articles, devices, compositions, systems, methods, and processes of the claimed invention encompass variations and adaptations developed using information from the embodiments described herein. Adaptation and/or modification of the articles, devices, compositions, systems, methods, and processes described herein may be performed, as contemplated by this description.

Throughout the description, where articles, devices, compositions, and systems are described as having, including, or comprising specific components, or where processes and methods are described as having, including, or comprising specific steps, it is contemplated that, additionally, there are articles, devices, compositions, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. The Background section is presented for purposes of clarity and is not meant as a description of prior art with respect to any claim.

Headers are provided for the convenience of the reader—the presence and/or placement of a header is not intended to limit the scope of the subject matter described herein.

### Compounds

The present disclosure encompasses the recognition that aliphatic carbonates (e.g., ROC(O)OR′) and aliphatic polycarbonates are useful constituents of electrolyte compositions for lithium ion batteries with many desirable attributes. However, such carbonates are unsuitable for lithium sulfur batteries in part because nucleophilic lithium sulfide intermediates are prone to react with the carbonate moiety leading to irreversible production of lithium alkoxides and formation of covalent carbon-sulfur linkages. Thus, the present disclosure includes a recognition of a previously unidentified source of a problem in the application of conventional electrolytes to lithium sulfur batteries.

The present disclosure describes, among other things, the use of thiocarbonates (e.g., trithiocarbonates or RSC(S)SR′) in place of traditional aliphatic carbonates as electrolytes in lithium sulfur cells. While not wishing to be bound by any particular theory, upon reaction with nucleophilic sulfides, trithiocarbonates do not form alkoxides and therefore the reaction can be reversible, and should thereby be less disruptive to the electrochemical cell operation.

By way of example, a first product of attack of polysulfide on a trithiocarbonate would be a tetrahedral intermediate arising from sulfide attack at the thiocarbonyl carbon:

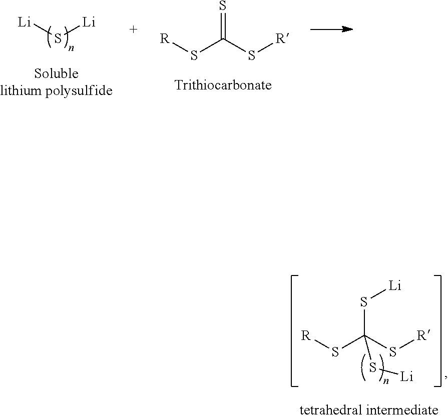

where, e.g., 8≥n≥3.

The resulting intermediate can collapse to reform the original starting material (i.e. release of [LiSₙ]⁻), or can expel [SR]⁻, or [SR′]⁻, leaving a thiocarbonate containing a lithiated polysulfide (RSC(S)SₙLi):

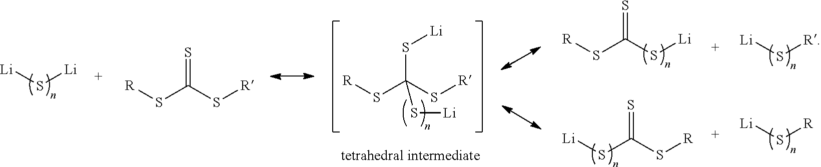

The released LiSR species are potent nucleophiles that can react with another trithiocarbonate such that, on balance, the composition will settle back to the starting trithiocarbonate mixture (with scrambling of substituents for non-symmetrically substituted trithiocarbonates) as the polysulfides are further reduced toward S²⁻. It will be appreciated that due to the potential scrambling of starting trithiocarbonates, the electrolyte composition in an operating or cycled battery may differ from the starting electrolyte composition. In addition, the existence of polysulfide substituted trithiocarbonates can change depending on state of battery charge—in this regard, both the relative abundance of these molecules and the average value of n therein (i.e. the length of polysulfide chains) may be dynamic.

Because irreversible side reactions can occur each cycle of an electrochemical cell, one or more performance advantages of electrochemical cells that include an electrolyte, additive, or binder as disclosed herein may be greater later in cycle life. For example, an electrochemical cell that includes an electrolyte, additive, or binder as disclosed herein may exhibit improved properties after at least 25, at least 50, at least 75, at least 100, at least 200, at least 300, or at least 500 charge cycles. For example, polysulfide migration may be mitigated by using a thiocarbonyl containing species (e.g., in a binder and/or electrolyte) and the effects of such mitigation may be more noticeable after many cycles than after only a few cycles. Mitigation of polysulfide migration may also be improved when an electrochemical cell includes a binder that comprises a thiocarbonyl functional group.

According to one aspect of the present disclosure, thiocarbonyl compounds are provided for use as a binder, electrolyte, or additive for an electrolyte in a lithium sulfur battery (e.g., a sulfur cathode). In some embodiments, the present disclosure provides a binder for a sulfur cathode comprising a thiocarbonyl functional group (i.e., —C(S)—). In some embodiments, the present disclosure provides an additive for an electrolyte in a lithium sulfur battery comprising a thiocarbonyl functional group. In some embodiments, the present disclosure provides an electrolyte for a lithium sulfur battery comprising a thiocarbonyl functional group.

In some embodiments, a provided binder, additive, or electrolyte comprises thiocarbonyl functional groups of formula X₂C═S (i.e., X—C(S)—X), where each X is independently selected from oxygen, nitrogen, sulfur, or carbon. In some embodiments, a provided binder, additive, or electrolyte comprises thiocarbonyl functional groups of formula X_{y}C═S, where each X is independently selected from oxygen, nitrogen, sulfur, or carbon, y is 1 or 2, and where one X is taken together with the other X and intervening atoms to form a ring, and when y is 1, X is connected to the thiocarbonyl carbon via a double bond. In some embodiments, y is 2 and a provided binder, additive, or electrolyte comprises thiocarbonyl functional groups of formula X₂C═S. In some embodiments, a provided binder, additive, or electrolyte comprises a thiocarbonate, thiourea, thiocarbamate, or thioketone functional group. In some embodiments, y is 1. In some embodiments, a provided binder, additive, or electrolyte comprises an isothiocyanate functional group. In some embodiments, a provided binder, additive, or electrolyte comprises carbon disulfide.

In some embodiments, a provided binder, additive, or electrolyte comprises thiocarbonyl functional groups of formula X_{y}C═S, where one X is nitrogen and the other X is independently selected from oxygen, nitrogen, sulfur, or carbon, and y is 2. In some embodiments, a provided binder, additive, or electrolyte comprises thiocarbonyl functional groups of formula X_{y}C═S, where one X is nitrogen and the other X is carbon, and y is 2. In some embodiments, a provided binder, additive, or electrolyte comprises thiocarbonyl functional groups of formula X_{y}C═S, where both X are nitrogen, and y is 2.

It will be appreciated that certain compounds useful as binders, additives, or electrolytes described herein may not comprise a thiocarbonyl functional group prior to a first cycling of secondary battery comprising such compound, but during cycling of the battery can in situ form a thiocarbonyl group either as a reaction product or intermediate.

In some embodiments, compounds comprising a thiocarbonyl functional group that may be used in accordance with the present disclosure are depicted below in Formulae I′, I, II, III, and IV, and classes and subclasses described herein, including species thereof (collectively, “provided compounds”).

In some embodiments, the present disclosure provides a compound of Formula I′:

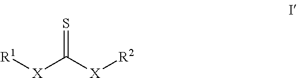

wherein R¹ and R² are each independently hydrogen or an optionally substituted group selected from C₁₋₁₅ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; or R¹ and R² are taken together with intervening atoms to form an optionally substituted ring;
each X is absent or independently selected from O, S, NR^{z}, and CR³R⁴;
each R^{z} is independently hydrogen or optionally substituted Ct-12 aliphatic;
each R³ and R⁴ is independently hydrogen, halogen, —CN, —NO₂, —N(R)₂, —OR, —SR, or an optionally substituted group selected from C₁₋₁₂ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
each R is independently hydrogen, optionally substituted C₁₋₆ aliphatic, optionally substituted 3- to 7-membered saturated or partially unsaturated carbocyclyl, or optionally substituted 3- to 7-membered saturated or partially unsaturated monocyclic heterocyclyl having 1-3 heteroatoms independently selected from nitrogen, oxygen, and sulfur, or two R when attached to the same nitrogen atom are taken together form an optionally substituted 3- to 7-membered saturated or partially unsaturated monocyclic heterocyclyl having 0-2 additional heteroatoms independently selected from nitrogen, oxygen, and sulfur.

In some embodiments, the present disclosure provides a compound of Formula I:

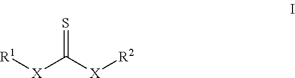

wherein R¹ and R² are each independently an optionally substituted group selected from C₁₋₁₅ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; or R¹ and R² are taken together with intervening atoms to form an optionally substituted ring;
each X is absent or independently selected from O, S, NR, and CR³R⁴;
each R^{z} is independently hydrogen or optionally substituted Ct-12 aliphatic;
each R³ and R⁴ is independently hydrogen, halogen, —CN, —NO₂, —N(R)₂, —OR, —SR, or an optionally substituted group selected from C₁₋₁₂ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
each R is independently hydrogen, optionally substituted C₁₋₆ aliphatic, optionally substituted 3- to 7-membered saturated or partially unsaturated carbocyclyl, or optionally substituted 3- to 7-membered saturated or partially unsaturated monocyclic heterocyclyl having 1-3 heteroatoms independently selected from nitrogen, oxygen, and sulfur, or two R when attached to the same nitrogen atom are taken together form an optionally substituted 3- to 7-membered saturated or partially unsaturated monocyclic heterocyclyl having 0-2 additional heteroatoms independently selected from nitrogen, oxygen, and sulfur.

In some embodiments, R¹ and R² are each independently hydrogen or an optionally substituted group selected from C₁₋₁₂ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

In some embodiments, R¹ and R² are each independently an optionally substituted group selected from C₁₋₁₂ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

In some embodiments, R¹ and R² are each independently an optionally substituted group selected from C₁₋₁₂ aliphatic. In some embodiments, R¹ and R² are each independently an optionally substituted group selected from C₁₋₆ aliphatic. In some embodiments, R¹ and R² are each independently an optionally substituted group selected from C₁₋₆ alkyl. In some embodiments, R¹ and R² are each independently an optionally substituted group selected from C₁₋₄ alkyl. In some embodiments, R¹ and R² are each independently methyl, ethyl, propyl, or butyl. In some embodiments, R¹ and R² are each independently substituted methyl. In some embodiments, R¹ and R² are each benzyl. In some embodiments, R and R² are each hydrogen.

In some embodiments, R¹ and R² are each independently an optionally substituted group selected from C₁₋₁₂ aliphatic or 3- to 7-membered saturated or partially unsaturated monocyclic carbocyclyl. In some embodiments, R¹ and R² are each independently an optionally substituted group selected from C₁₋₆ aliphatic or 3- to 4-membered saturated monocyclic carbocyclyl.

In some embodiments, R¹ and R² are taken together with intervening atoms to form an optionally substituted ring selected from 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

In some embodiments, R¹ and R² are taken together with intervening atoms to form an optionally substituted 3- to 7-membered saturated or partially unsaturated monocyclic carbocyclyl. In some embodiments, R¹ and R² are taken together with intervening atoms to form an optionally substituted 4- to 10-membered saturated or partially unsaturated bicyclic carbocyclyl. In some embodiments, R¹ and R² are taken together with intervening atoms to form an optionally substituted 3- to 7-membered saturated or partially unsaturated monocyclic heterocyclyl having 1-3 heteroatoms independently selected from nitrogen, oxygen, and sulfur. In some embodiments, R¹ and R² are taken together with intervening atoms to form an optionally substituted 7- to 10-membered saturated or partially unsaturated bicyclic heterocyclyl having 1-4 heteroatoms independently selected from nitrogen, oxygen, and sulfur. In some embodiments, R¹ and R² are taken together with intervening atoms to form an optionally substituted phenyl. In some embodiments, R¹ and R² are taken together with intervening atoms to form an optionally substituted 8- to 10-membered bicyclic aryl. In some embodiments, R¹ and R² are taken together with intervening atoms to form an optionally substituted 5- to 6-membered monocyclic heteroaryl having 1-4 heteroatoms independently selected from nitrogen, oxygen, and sulfur. In some embodiments, R¹ and R² are taken together with intervening atoms to form an optionally substituted 8- to 10-membered bicyclic heteroaryl having 1-4 heteroatoms independently selected from nitrogen, oxygen, and sulfur.

In some embodiments, R¹ and R² are taken together with intervening atoms to form an optionally substituted 3- to 7-membered saturated or partially unsaturated monocyclic heterocyclyl having 1-3 heteroatoms independently selected from nitrogen, oxygen, and sulfur. In some embodiments, R¹ and R² are taken together with intervening atoms to form an optionally substituted 5-membered saturated or partially unsaturated monocyclic heterocyclyl having 1-3 heteroatoms independently selected from nitrogen, oxygen, and sulfur. In some embodiments, R¹ and R² are taken together with intervening atoms to form an optionally substituted 5-membered saturated or partially unsaturated monocyclic heterocyclyl having 2 heteroatoms independently selected from nitrogen, oxygen, and sulfur. In some embodiments, R¹ and R² are taken together with intervening atoms to form an optionally substituted 5-membered saturated or partially unsaturated monocyclic heterocyclyl having 2 sulfur heteroatoms.

In some embodiments, R¹ and R² are taken together with intervening atoms to form an optionally substituted 5- to 6-membered monocyclic heteroaryl having 1-4 heteroatoms independently selected from nitrogen, oxygen, and sulfur. In some embodiments, R¹ and R² are taken together with intervening atoms to form an optionally substituted 5-membered monocyclic heteroaryl having 2-3 heteroatoms independently selected from nitrogen, oxygen, and sulfur. In some embodiments, R¹ and R² are taken together with intervening atoms to form an optionally substituted 6-membered monocyclic heteroaryl having 2-3 heteroatoms independently selected from nitrogen, oxygen, and sulfur. In some embodiments, R¹ and R² are taken together with intervening atoms to form an optionally substituted 6-membered monocyclic heteroaryl having 3 nitrogen heteroatoms. In some embodiments, R¹ and R² are taken together with intervening atoms to form an optionally substituted triazine. In some embodiments, R¹ and R² are taken together with intervening atoms to form an optionally substituted 1,3,5-triazine.

In some embodiments, R¹ and R² are taken together with intervening atoms to form Ring A as in Formula II:

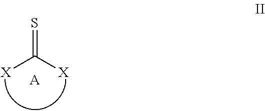

wherein X is as defined above for Formula I and described in classes and subclasses herein, both singly and in combination, and
Ring A is an optionally substituted ring selected from 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

In some embodiments, Ring A is optionally substituted 3- to 7-membered saturated or partially unsaturated monocyclic heterocyclyl having 1-3 heteroatoms independently selected from nitrogen, oxygen, and sulfur. In some embodiments, Ring A is optionally substituted 5-membered saturated or partially unsaturated monocyclic heterocyclyl having 1-3 heteroatoms independently selected from nitrogen, oxygen, and sulfur. In some embodiments, Ring A is optionally substituted 5-membered saturated or partially unsaturated monocyclic heterocyclyl having 2 heteroatoms independently selected from nitrogen, oxygen, and sulfur. In some embodiments, Ring A is optionally substituted 5-membered saturated or partially unsaturated monocyclic heterocyclyl having 2 sulfur heteroatoms.

In some embodiments, Ring A is optionally substituted 5- to 6-membered monocyclic heteroaryl having 1-4 heteroatoms independently selected from nitrogen, oxygen, and sulfur. In some embodiments, Ring A is optionally substituted 5-membered monocyclic heteroaryl having 2-3 heteroatoms independently selected from nitrogen, oxygen, and sulfur. In some embodiments, Ring A is optionally substituted 6-membered monocyclic heteroaryl having 2-3 heteroatoms independently selected from nitrogen, oxygen, and sulfur. In some embodiments, Ring A is 6-membered monocyclic heteroaryl having 3 nitrogen heteroatoms. In some embodiments, Ring A is optionally substituted triazine. In some embodiments, Ring A is optionally substituted 1,3,5-triazine.

As described above, all tautomeric forms of provided compounds are within the scope of the disclosure. It will therefore be appreciated that certain compounds comprising a thiol (i.e., —SH) functional group are within the scope of provided compounds and genera if a thiocarbonyl functional group is a tautomer. For example, a tautomer of trithiocyanuric acid comprises a thiocarbonyl (e.g., thiourea) functional group as shown below:

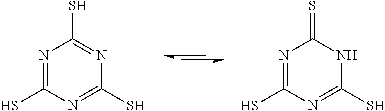 [SMILES: Sc1nc(S)nc(S)n1 ; S=c1nc(S)nc(S)[nH]1]

In some embodiments, both X are absent. In some embodiments, one X is absent. In some embodiments, each X is independently selected from O, S, NR², and CR³R⁴. In some embodiments, both X are S. In some embodiments, both X are NR². In some embodiments, both X are O. In some embodiments, both X are CR³R⁴. In some embodiments, one X is S and the other X is O. In some embodiments, one X is S and the other X is NR². In some embodiments, one X is S and the other X is CR³R⁴. In some embodiments, one X is S and the other X is absent.

In some embodiments, one X is O and the other X is NR². In some embodiments, one X is O and the other X is CR³R⁴. In some embodiments, one X is O and the other X is absent.

In some embodiments, one X is NR² and the other X is CR³R⁴. In some embodiments, one X is NR² and the other X is absent. In some embodiments, one X is CR³R⁴ and the other X is absent.

In some embodiments, R^{z} is hydrogen. In some embodiments, R¹ is optionally substituted C₁₋₁₂ aliphatic. In some embodiments, R^{z} is methyl.

In some embodiments, R³ is hydrogen, halogen, —CN, —NO₂, —N(R)₂, —OR, —SR, or an optionally substituted group selected from C₁₋₁₂ aliphatic. In some embodiments, R⁴ is hydrogen, halogen, —CN, —NO₂, —N(R)₂, —OR, —SR, or an optionally substituted group selected from C₁₋₁₂ aliphatic.

In some embodiments, each R is independently hydrogen, optionally substituted C₁₋₆ aliphatic, or two R when attached to the same nitrogen atom are taken together form an optionally substituted 3- to 7-membered saturated or partially unsaturated monocyclic heterocyclyl having 0-2 additional heteroatoms independently selected from nitrogen, oxygen, and sulfur. In some embodiments, each R in hydrogen or C₁₋₆ aliphatic.

In some embodiments, one X is NR^{z} and the other X is independently selected from O, S, NR^{z}, and CR³R⁴. In some embodiments, one X is NR, the other X is independently selected from O, S, NR, and CR³R⁴, and each of R^{z}, R¹, R², R³, and R⁴ is independently hydrogen or C₁₋₆ aliphatic. In some embodiments, one X is NR^{z}, the other X is independently selected from O, S, NR^{z}, and CR³R⁴, and each of R^{z}, R¹, R², R³, and R⁴ is hydrogen.

In some embodiments, each X is independently NR^{z}, and each of R^{z}, R¹, and R² is independently hydrogen or C₁₋₆ aliphatic. In some embodiments, each X is independently NR, and each of R^{z}, R¹, and R² is hydrogen.

In some embodiments, a provided compound is selected from ethylene trithiocarbonate and trithiocyanuric acid. In some embodiments, a provided compound is ethylene trithiocarbonate. In some embodiments, a provided compound is trithiocyanuric acid.

In some embodiments, a provided compound is 3H-1,2-benzodithiol-3-one. In some embodiments, a provided compound is phenylacetyl disulfide. In some embodiments, a provided compound is tetramethylthiourea. In some embodiments, a provided compound is thioacetamide. In some embodiments, a provided compound is thiourea. In some embodiments, a provided compound is trithiocyanuric acid. In some embodiments, a provided compound is vinylene trithiocarbonate. In some embodiments, a provided compound is zinc dimethyldithiocarbamate. In some embodiments, a provided compound is dimethyl trithiocarbonate.

In some embodiments, a provided compound is or comprises a disulfide moiety that forms a thiocarbonyl moiety as a reaction product or intermediate upon cycling of a secondary battery comprising such compound as a binder, additive, or electrolyte.

In some embodiments, a provided compound is selected from a compound in Table 1.

TABLE 1

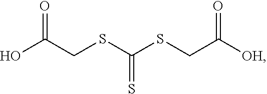 [SMILES: O=C(O)CSC(=S)SCC(=O)O]

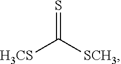

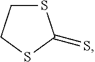

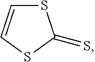

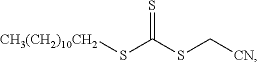

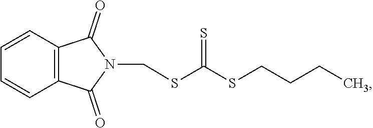 [SMILES: CCCCSC(=S)SCN1C(=O)c2ccccc2C1=O]

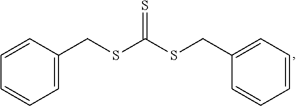 [SMILES: S=C(SCc1ccccc1)SCc1ccccc1]

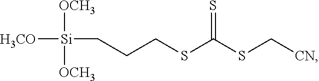 [SMILES: CO[Si](CCCSC(=S)SCC#N)(OC)OC]

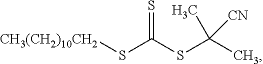

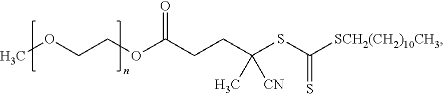

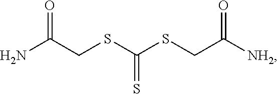 [SMILES: NC(=O)CSC(=S)SCC(N)=O]

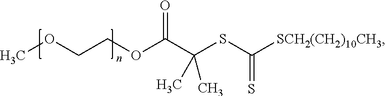

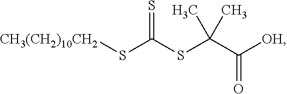

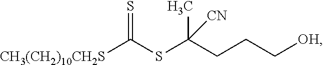

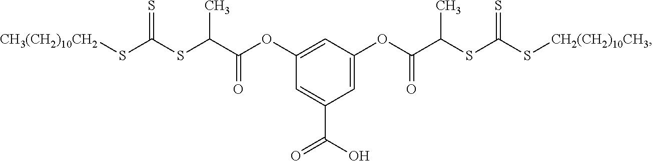

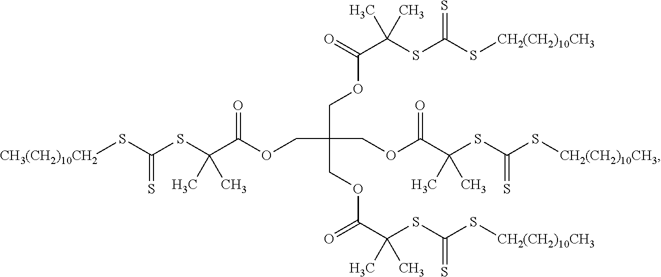

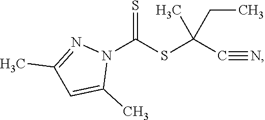 [SMILES: CCC(C)(C#N)SC(=S)n1nc(C)cc1C]

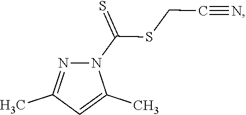

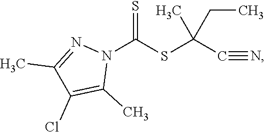 [SMILES: CCC(C)(C#N)SC(=S)n1nc(C)c(Cl)c1C]

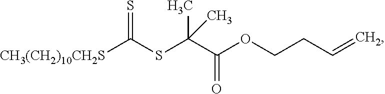

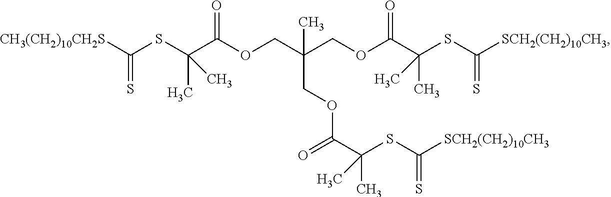

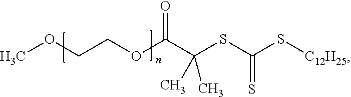

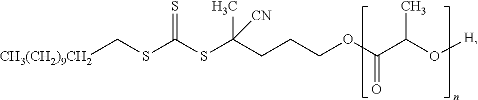

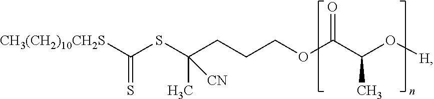

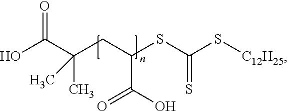

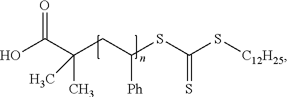

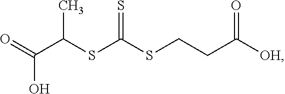

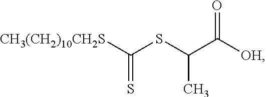

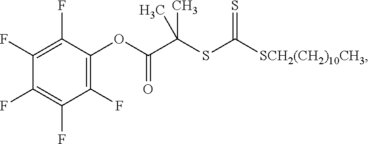

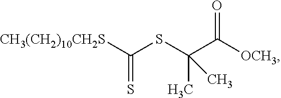

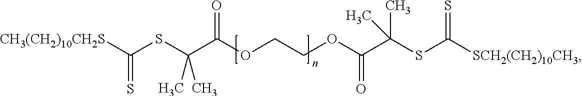

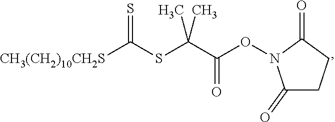

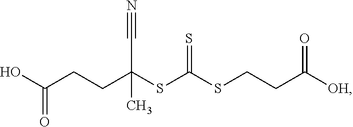 [SMILES: CC(C#N)(CCC(=O)O)SC(=S)SCCC(=O)O]

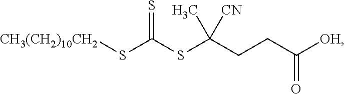

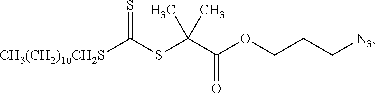

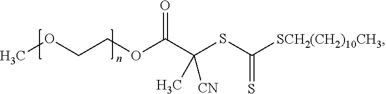

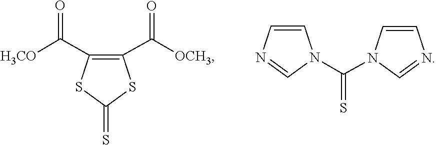 [SMILES: COC(=O)c1sc(=S)sc1C(=O)OC ; S=C(n1ccnc1)n1ccnc1]

In some embodiments, a provided compound is other than dimethyl trithiocarbonate. In some embodiments, a provided compound is other than ethylene trithiocarbonate.

In some embodiments, a binder, additive, or electrolyte comprises a thiocarbonyl functional group and a moiety comprising a carbon atom bonded to both a nitrogen and sulfur atom (“NCS moiety”). In some embodiments, a NCS moiety comprises at least a portion of the thiocarbonyl functional group (e.g., the carbon bonded to sulfur is a thiocarbonyl group).

### Electrolyte Compositions

As described above, an electrolyte of the present disclosure can comprise thiocarbonyl functional groups, including compounds of any one of Formulae I′, I, II, III, or IV.

While not wishing to be bound by any particular theory, upon cycling of a secondary battery comprising an electrolyte of Formula I, a polysulfide attacks the thiocarbonyl group and upon release of a lithium-X (e.g., lithium thiolate) species, the electrolyte compound is transformed into a thiocarbonyl-containing compound that comprises a lithiated polysulfide. Thus, in certain embodiments a provided electrolyte composition comprises a compound of Formula III:

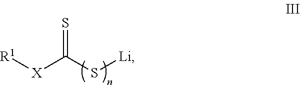

wherein n is 3, 4, 5, 6, 7, or 8.

In addition, in the case of asymmetric thiocarbonyl compounds, other lithiated polysulfide compounds can be formed depending upon which group(s) are released after polysulfide attack. Thus, in certain embodiments a provided electrolyte composition comprises a compound of Formula IV:

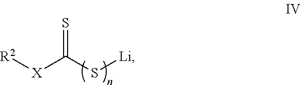

wherein n is 3, 4, 5, 6, 7, or 8.

In some embodiments, where at least one X is sulfur, electrolyte compositions further comprise Li(S)ₙR¹ and/or Li(S)ₙR².

In some embodiments, a provided electrolyte composition is “uncycled”, meaning it has not yet been subjected to a charge and/or discharge.

Electrolyte compositions of the present disclosure may also comprise other electrolytes or components, including those described below.

In certain embodiments, a secondary sulfur battery comprises an electrolyte comprising an electrolytic salt. Examples of electrolytic salts include, for example, lithium trifluoromethane sulfonimide, lithium triflate, lithium perchlorate, LiPF₆, LiBF₄, tetraalkylammonium salts (e.g. tetrabutylammonium tetrafluoroborate, TBABF₄), liquid state salts at room temperature (e.g. imidazolium salts, such as 1-ethyl-3-methylimidazolium bis-(perfluoroethyl sulfonyl)imide, EMIBeti), and the like. In some embodiments, lithium trithiocarbonate(s) is the primary lithium salt in the electrolyte composition.

In certain embodiments, an electrolyte comprises one or more alkali metal salts. In certain embodiments, such salts comprise lithium salts, such as LiCF₃SO₃, LiClO₄, LiNO₃, LiPF₆, LiBr, LiTDI, LiFSI, and LiTFSI, or combinations thereof. In certain embodiments, an electrolyte comprises ionic liquids, such as 1-ethyl-3-methylimidzaolium-TFSI, N-butyl-N-methyl-piperidinium-TFSI, N-methyl-n-butyl pyrrolidinium-TFSI, and N-methyl-N-propylpiperidinium-TFSI, or combinations thereof. In certain embodiments, an electrolyte comprises superionic conductors, such as sulfides, oxides, and phosphates, for example, phosphorous pentasulfide, or combinations thereof.

In certain embodiments, an electrolyte is a liquid. For example, in certain embodiments, an electrolyte comprises an organic solvent. In certain embodiments, an electrolyte comprises only one organic solvent. In some embodiments, an electrolyte comprises a mixture of two or more organic solvents. In certain embodiments, a mixture of organic solvents comprising one or more weak polar solvents, strong polar solvents, and lithium protecting solvents.

The term “weak polar solvent”, as used herein, is defined as a solvent that is capable of dissolving elemental sulfur and has a dielectric coefficient of less than 15. A weak polar solvent is selected from aryl compounds, bicyclic ethers, and acyclic carbonate compounds. Examples of weak polar solvents include xylene, dimethoxyethane, 2-methyltetrahydrofuran, diethyl carbonate, dimethyl carbonate, toluene, dimethyl ether, diethyl ether, diglyme, tetraglyme, and the like. The term “strong polar solvent”, as used herein, is defined as a solvent that is capable of dissolving lithium polysulfide and has a dielectric coefficient of more than 15. A strong polar solvent is selected from bicyclic carbonate compounds, sulfoxide compounds, lactone compounds, ketone compounds, ester compounds, sulfate compounds, and sulfite compounds. Examples of strong polar solvents include hexamethyl phosphoric triamide, γ-butyrolactone, acetonitrile, ethylene carbonate, propylene carbonate, N-methylpyrrolidone, 3-methyl-2-oxazolidone, dimethyl formamide, sulfolane, dimethyl acetamide, dimethyl sulfoxide, dimethyl sulfate, ethylene glycol diacetate, dimethyl sulfite, ethylene glycol sulfite, and the like. The term “lithium protection solvent”, as used herein, is defined as a solvent that forms a good protective layer, i.e. a stable solid-electrolyte interface (SEI) layer, on a lithium surface, and which shows a cyclic efficiency of at least 50%. A lithium protection solvent is selected from saturated ether compounds, unsaturated ether compounds, and heterocyclic compounds including one or more heteroatoms selected from the group consisting of N, O, and/or S. Examples of lithium protection solvents include tetrahydrofuran, 1,3-dioxolane, 3,5-dimethylisoxazole, 2,5-dimethyl furan, furan, 2-methyl furan, 1,4-oxane, 4-methyldioxolane, and the like.

In certain embodiments, an electrolyte is a liquid (e.g., an organic solvent). In some embodiments, a liquid is selected from the group consisting of organocarbonates, ethers, sulfones, water, alcohols, fluorocarbons, or combinations of any of these. In certain embodiments, an electrolyte comprises an ethereal solvent.

In certain embodiments, an organic solvent comprises an ether. In certain embodiments, an organic solvent is selected from the group consisting of 1,3-dioxolane, dimethoxyethane, diglyme, triglyme, γ-butyrolactone, γ-valerolactone, and combinations thereof. In certain embodiments, an organic solvent comprises a mixture of 1,3-dioxolane and dimethoxyethane. In certain embodiments, an organic solvent comprises a 1:1 v/v mixture of 1,3-dioxolane and dimethoxyethane. In certain embodiments, an organic solvent is selected from the group consisting of: diglyme, triglyme, γ-butyrolactone, γ-valerolactone, and combinations thereof. In certain embodiments, an electrolyte comprises sulfolane, sulfolene, dimethyl sulfone, methyl ethyl sulfone, or a combination thereof. In some embodiments, an electrolyte comprises ethylene carbonate, propylene carbonate, dimethyl carbonate, diethyl carbonate, methylethyl carbonate, or a combination thereof.

In certain embodiments, an electrolyte is a solid. In certain embodiments, a solid electrolyte comprises a polymer. In certain embodiments, a solid electrolyte comprises a glass, a ceramic, an inorganic composite, or combinations thereof. In certain embodiments, a solid electrolyte comprises a polymer composite with a glass, a ceramic, an inorganic composite, or combinations thereof. In certain embodiments, such solid electrolytes comprise one or more liquid components as plasticizers or to form a “gel electrolyte”.

### Cathode

Cathode materials of the present disclosure have utility in manufacture of electrochemical devices. They may be porous or non-porous. Certain compositions disclosed herein would be adhered to a current collector to form cathodes for secondary sulfur batteries. Provided cathode compositions may comprise one or more additives such as electrically conductive particles, binders, and other functional additives typically found in battery cathode mixtures. Generally, provided compositions include plentiful conductive particles to increase electrical conductivity of a cathode and provide a low resistance pathway for electrons to access such manufactured cathode. In various embodiments, other additives are included in the composition to alter or otherwise enhance a cathode produced according to the principles described herein. Other cathode components include, for example, a current collector, connecting tabs, and the like.

In certain embodiments, the cathode composition includes a sulfur electroactive material (e.g., sulfur in its Ss cyclic octatomic molecular form) and/or in lithium sulfide (e.g., Li₂S₂ and/or Li₂S) and/or in the form of an electroactive organosulfur compound or an electroactive sulfur containing polymer. In certain embodiments, the electroactive material is an intercalation material structured to intercalate lithium ions. In certain embodiments, the electroactive material operates in a voltage range overlapping with the discharge voltage range of S₈→Li₂S (sulfur to lithium sulfide conversion), e.g., from about 1.8V to about 2.6V vs. Li⁰, e.g., from about 2.0V to about 2.4V vs. Li⁰.

In certain embodiments, the cathode composition contains conductive materials and a binder. In certain embodiments, a conductive material comprises an electrically conductive material that facilitates movement of electrons within a composite. For example, in certain embodiments, a conductive material is selected from the group consisting of carbon-based materials, graphite-based materials, conductive polymers, metals, semiconductors, metal oxides, metal sulfides, and combinations thereof. In certain embodiments, a conductive material comprises a carbon-based material. In certain embodiments, a conductive material comprises a graphite-based material. In certain embodiments, the cathode composition does not contain carbon, or contains a low amount of carbon (e.g., no greater than 5.0 wt. %, no greater than 3.0 wt. %, no greater than 2.0 wt. %, no greater than 1.0 wt. %, or no greater than 0.5 wt. %).

In certain embodiments, an electrically conductive material is selected from the group consisting of conductive carbon powders, such as carbon black, Super P®, C-NERGY™ Super C65, Ensaco® black, Ketjenblack®, acetylene black, synthetic graphite such as Timrex® SFG-6, Timrex® SFG-15, Timrex® SFG-44, Timrex® KS-6, Timrex® KS-15, Timrex® KS-44, natural flake graphite, carbon nanotubes, fullerenes, hard carbon, mesocarbon microbeads, and the like. In certain embodiments, a conductive material comprises one or more conductive polymers. For example, in certain embodiments, a conductive polymer is selected from the group consisting of polyaniline, polythiophene, polyacetylene, polypyrrole, and the like. In some embodiments, a conductive polymer is a cationic polymer. In some embodiments, a cationic polymer is a quaternary ammonium polymer. In certain embodiments, a cationic polymer is selected from the group consisting of a polydiallyldimethylammonium salt, a poly[(3-chloro-2-hydroxypropyl)methacryloxyethyldimethyl-ammonium salt, a poly(butyl acrylate-methacryloxyethyltrimethylammonium) salt, poly(1-methyl-4-vinylpyridinium) salt, a poly(1-methyl-2-vinylpyridinium) salt, and a poly(methyacryloxyethyltriethylammonium) salt. In certain embodiments, a cationic polymer is selected from polydiallyldimethylammonium chloride (polyDADMAC), polybrene, epichlorohydrin-dimethylamine (epi-DMA), poly[(3-chloro-2-hydroxypropyl)methacryloxyethyldimethyl-ammonium chloride), poly(acrylamide-methacryloxyethyltrimethylammonium bromide), poly(butyl acrylate-methacryloxyethyltrimethylammonium bromide), poly(1-methyl-4-vinylpyridinium bromide), poly(1-methyl-2-vinylpyridinium bromide), and poly(methyacryloxyethyltriethylammonium bromide). In certain embodiments, a conductive material comprises one or more metal oxides or sulfides. For example, in certain embodiments, a conductive material comprises one or more oxides or sulfides of a first-row transition metal such as titanium, vanadium, chromium, manganese, iron, cobalt, copper, zinc, or combinations thereof. For example, in certain embodiments, a conductive material comprises one or more oxides or sulfides of a second-row transition metal such as zirconium, indium, tin, antimony, or combinations thereof. In certain embodiments, a conductive material is used alone. In other embodiments, a conductive material is used as a mixture of two or more conductive materials described above.

In certain embodiments, a binder is included in the provided cathode composition materials. Binders are generally polymeric materials that help adhere individual particles composing a cathode mixture into a stable composite. Representative binders include polyvinylidene fluoride, poly(vinylidene fluoride-co-hexafluoropropene) (PVDF/HFP), polytetrafluoroethylene (PTFE), Kynar Flex® 2801, Kynar® Powerflex LBG, Kynar® HSV 900, Teflon®, carboxymethylcellulose, styrene-butadiene rubber (SBR), polyethylene oxide, polypropylene oxide, polyethylene, polypropylene, polyacrylates, polyvinyl pyrrolidone, poly(methyl methacrylate), polyethyl acrylate, polytetrafluoroethylene, polyvinyl chloride, polyacrylonitrile, polycaprolactam, polyethylene terephthalate, polybutadiene, polyisoprene or polyacrylic acid, or derivatives, mixtures, or copolymers of any of these. In some embodiments, a binder is water soluble binder, such as sodium alginate, carrageenan, or carboxymethyl cellulose. Generally, binders hold active materials together and in contact with a current collector (e.g., a metal foil such as aluminum, stainless steel, or copper, or a conductive carbon sheet). In certain embodiments, a binder is selected from the group consisting of poly(vinyl acetate), polyvinyl alcohol, polyethylene oxide, polyvinyl pyrrolidone, alkylated polyethylene oxide, crosslinked polyethylene oxide, polyvinyl ether, poly(methyl methacrylate), polyvinylidene fluoride, a copolymer of polyhexafluoropropylene and polyvinylidene fluoride, polyethyl acrylate, polytetrafluoroethylene, polyvinyl chloride, polyacrylonitrile, polyvinyl pyridine, polystyrene, and derivatives, mixtures, and copolymers thereof. In some embodiments, a binder is a cationic polymer. In some embodiments, a binder is a quaternary ammonium polymer. In some embodiments, a binder is a cationic polymer as described above.

In some embodiments, the cathode composition further comprises a binder comprising a thiocarbonyl group (e.g., a provided compound). In some embodiments, the cathode composition further comprises a binder comprising a compound of any one of Formulae I, II, III, or IV.

In certain embodiments, a cathode further comprises a coating layer. For example, in certain embodiments, a coating layer comprises a polymer, an organic material, an inorganic material, or a mixture thereof that is not an integral part of the porous composite or the current collector. In certain such embodiments, a polymer is selected from the group consisting of polyvinylidene fluoride, a copolymer of polyvinylidene fluoride and hexafluoropropylene, poly(vinyl acetate), poly(vinyl butyral-co-vinyl alcohol-co-vinyl acetate), poly(methylmethacrylate-coethyl acrylate), polyacrylonitrile, polyvinyl chloride-co-vinyl acetate, polyvinyl alcohol, poly(1-vinylpyrrolidone-covinyl acetate), cellulose acetate, polyvinyl pyrrolidone, polyacrylate, polymethacrylate, polyolefin, polyurethane, polyvinyl ether, acrylonitrile-butadiene rubber, styrenebutadiene rubber, acrylonitrile-butadiene styrene, a sulfonated styrene/ethylene-butylene/styrene triblock copolymer, polyethylene oxide, and derivatives, mixtures, and copolymers thereof. In some embodiments, a coating layer comprises a cationic polymer. In some embodiments, a coating layer comprises a quaternary ammonium polymer. In some embodiments, a coating layer comprises a cationic polymer as described above. In certain such embodiments, an inorganic material comprises, for example, colloidal silica, amorphous silica, surface-treated silica, colloidal alumina, amorphous alumina, tin oxide, titanium oxide, titanium sulfide (TiS₂), vanadium oxide, zirconium oxide (ZrO₂), iron oxide, iron sulfide (FeS), iron titanate (FeTiO₃), barium titanate (BaTiO₃), and combinations thereof. In certain embodiments, an organic material comprises conductive carbon.

Suitable materials for use in cathode mixtures are disclosed in Cathode Materials for Lithium Sulfur Batteries: Design, Synthesis, and Electrochemical Performance, Lianfeng, et al., Interchopen.com, Published June 1st 2016, and The strategies of advanced cathode composites for lithium-sulfur batteries, Zhou et al., SCIENCE CHINA Technological Sciences, Volume 60, Issue 2: 175-185(2017), the entire disclosures of each of which are hereby incorporated by reference herein.

In certain embodiments, the cathode comprises one or more of the following features: (a) a “stack” of multi-functional materials (e.g., wherein the stack comprises, for example, particles with gradient structures that balance the transport of ions and electrons for improved power capability, energy density, and life; bi-functional cathode additives that simultaneously store Li and conduct electrons, replacing expensive and space-wasting carbons; a binding molecule that spatially constrains the electrochemical reaction storing the energy and thereby extends life; electrolyte components that improve the basic efficiency of the electrolyte, providing improved energy density; and/or a cathode design that enables greater safety and energy density); (b) a tight electrode layer; (c) a tight tertiary structure; (d) porosity control; (e) a core-shell structure; (f) a cross-linked polymer shell; (g) a self-doped polymer shell; (h) an ion conductive binder; (i) a dual layer hybrid cathode; (j) a polymer that traps polysulfide; (k) a three-dimensional structure with high surface area (e.g., to hold both carbon and lithium, e.g., to intercalate); and (1) a three-dimensional structure within which carbon is replaced with a metal disulfide (e.g., and wherein the battery comprises a polymer electrolyte for sulfur).

### Anode

In certain embodiments, a secondary sulfur battery comprises a lithium anode. A lithium anode suitable for use in lithium-sulfur cells may be used. In certain embodiments, an anode of a secondary sulfur battery comprises a negative active material selected from materials in which lithium intercalation reversibly occurs, materials that react with lithium ions to form a lithium-containing compound, metallic lithium, lithium alloys, and combinations thereof. In certain embodiments, an anode comprises metallic lithium. In certain embodiments, lithium-containing anodic compositions comprise carbon-based compounds. In certain embodiments, a carbon-based compound is selected from the group consisting of crystalline carbon, amorphous carbon, graphite, and mixtures thereof. In certain embodiments, the anode does not contain carbon, or contains a low amount of carbon (e.g., no greater than 5.0 wt. %, no greater than 3.0 wt. %, no greater than 2.0 wt. %, no greater than 1.0 wt. %, or no greater than 0.5 wt. %). In certain embodiments, a material that reacts with lithium ions to form a lithium-containing compound is selected from the group consisting of tin oxide (SnO₂), titanium nitrate, and silicon. In certain embodiments, a lithium alloy comprises an alloy of lithium with another alkali metal (e.g. sodium, potassium, rubidium or cesium). In certain embodiments, a lithium alloy comprises an alloy of lithium with a transition metal. In certain embodiments, lithium alloys include alloys of lithium and a metal selected from the group consisting of Na, K, Rb, Cs, Fr, Be, Mg, Ca, Sr, Ba, Ra, Al, Sn, In, Zn, Sm, La, and combinations thereof. In certain embodiments, a lithium alloy comprises an alloy of lithium with indium. In certain embodiments, a lithium alloy comprises an alloy of lithium with aluminum. In certain embodiments, a lithium alloy comprises an alloy of lithium with zinc. In certain embodiments, an anode comprises a lithium-silicon alloy. Examples of suitable lithium-silicon alloys include: Li₁₅Si₄, Li₁₂Si₇, Li₇Si₃, Li₁₃Si₄, and Li₂₁Si₅/Li₂₂Si₅. In certain embodiments, a lithium metal or lithium alloy is present as a composite with another material. In certain embodiments, such composites include materials such as graphite, graphene, metal sulfides or oxides, or conductive polymers.

In some embodiments, an anode is protected against redox shuttling reactions and hazardous runaway reactions by any of the methodologies reported in the art, for example, by creating a protective layer on a surface of an anode by chemical passivation or by deposition or polymerization. For example, in certain embodiments, an anode comprises an inorganic protective layer, an organic protective layer, or a mixture thereof, on a surface of lithium metal. In certain embodiments, an inorganic protective layer comprises Mg, Al, B, Sn, Pb, Cd, Si, In, Ga, lithium silicate, lithium borate, lithium phosphate, lithium phosphoronitride, lithium silicosulfide, lithium borosulfide, lithium aluminosulfide, lithium phosphosulfide, lithium fluoride or combinations thereof. In certain embodiments, an organic protective layer includes a conductive monomer, oligomer, or polymer. In certain embodiments, such polymer is selected from poly(p-phenylene), polyacetylene, poly(p-phenylene vinylene), polyaniline, polypyrrole, polythiophene, poly(2,5-ethylene vinylene), acetylene, poly(perinaphthalene), polyacene, and poly(naphthalene-2,6-di-y1), or combinations thereof.

Moreover, in certain embodiments, inactive sulfur material, generated from an electroactive sulfur material of a cathode, during charging and discharging of a secondary sulfur battery, attaches to an anode surface. The term “inactive sulfur”, as used herein, refers to sulfur that cannot participate in an electrochemical reaction of a cathode such that it contributes no capacity upon repeated charge/discharge cycles. In certain embodiments, inactive sulfur on an anode surface acts as a protective layer on such anode. In certain embodiments, inactive sulfur is present in the form of lithium sulfide.

It is further contemplated that the concepts of the present disclosure can be adapted for use in sodium-sulfur batteries. Such sodium-sulfur batteries comprise a sodium-based anode and an intercalation or conversion material capable of intercalating or reacting with sodium ions. Such systems are encompassed within embodiments of the present disclosure.

It is further contemplated that the present disclosure can be adapted for use in batteries constructed in an anode-free configuration. In certain embodiments, a manufactured battery or battery component has an anode-free configuration and comprises an anodic current collector (e.g. copper) and one or more of the following: (a) a thin layer of garnet, (b) a structure (e.g., a complex 3D structure) with a coating deposited by atomic layer deposition (ALD) (e.g., wherein the ALD coating comprises one or more members selected from the group consisting of lithium phosphorus oxynitride (LiPON), garnet, an oxide, perovskite, a sulphide, Li₃BO₃—Li₂CO₃ (LBCO), a sodium super ionic conductor (NASICON), and alumina); (c) a polymer (e.g., polyethylene oxide (PEO) or a block copolymer); (d) lithium phosphorus oxynitride (LiPON), and (e) a solid-electrolyte interface (SEI) layer (e.g., an artificial SEI layer formed in situ).

### Preparation of Electrodes

There are a variety of methods for manufacturing electrodes for use in a secondary sulfur battery. One such process, commonly referred to as a “wet process,” involves adding the solid cathode materials to a liquid to prepare a slurry composition. These slurries are typically in the form of a viscous liquid that is formulated to facilitate a downstream coating operation. A thorough mixing of a slurry can be important for coating and drying operations, which affect performance and quality of an electrode. Suitable mixing devices include ball mills, magnetic stirrers, sonication, planetary mixers, high speed mixers, homogenizers, universal type mixers, and static mixers. A liquid used to make a slurry can be any capable of homogeneously dispersing an active material, a binder, a conducting material, and any additives, and that is also able to be evaporated. Suitable slurry liquids include, for example, N-methylpyrrolidone, acetonitrile, methanol, ethanol, propanol, butanol, tetrahydrofuran, water, isopropyl alcohol, dimethylpyrrolidone, propylene carbonate, gamma butyrolactone and the like.

In some embodiments, a prepared composition is coated on a current collector and dried to form an electrode. Specifically, a slurry is used to coat an electrical conductor to form an electrode by evenly spreading a slurry on to a conductor, which is then, in certain embodiments, optionally roll-pressed (e.g. calendared) and/or heated as is known in the art. Generally, a matrix of an active material and conductive material are held together and on a conductor by a binder. In certain embodiments, a matrix comprises a polymer binder, such as polyvinylidene fluoride (PVDF), poly(vinylidene fluoride-co-hexafluoropropene) (PVDF/HFP), polytetrafluoroethylene (PTFE), Kynar Flex® 2801, Kynar® Powerflex LBG, Kynar® HSV 900, Teflon®, styrene butadiene rubber (SBR), polyethylene oxide (PEO), or polytetrafluoroethylene (PTFE). In certain embodiments, additional carbon particles, carbon nanofibers, carbon nanotubes, are dispersed in a matrix to improve electrical conductivity. Alternatively or additionally, in certain embodiments, lithium salts are dispersed in a matrix to improve lithium conductivity.

In certain embodiments, a current collector is selected from the group consisting of: aluminum foil, copper foil, nickel foil, stainless steel foil, titanium foil, zirconium foil, molybdenum foil, nickel foam, copper foam, carbon paper or carbon fiber sheets, polymer substrates coated with conductive metal, and/or combinations thereof.

PCT Publication Nos. WO2015/003184, WO2014/074150, and WO2013/040067, the entire disclosures of which are hereby incorporated by reference herein, describe various methods of fabricating electrodes and electrochemical cells.

### Separator

In certain embodiments, a secondary sulfur battery comprises a separator, which divides the anode and cathode and prevents direct electron conduction between them. In certain embodiments, the separator has a high lithium ion permeability. In certain embodiments, a separator is relatively less permeable to polysulfide ions dissolved in electrolyte. In certain such embodiments, a separator as a whole inhibits or restricts passage of electrolyte-soluble sulfides between anodic and cathodic portions of a battery. In certain embodiments, a separator of impermeable material is configured to allow lithium ion transport between anode and cathode of a battery during charging and discharging of a cell. In some such embodiments, a separator is porous. One or more electrolyte-permeable channels bypassing, or penetrating through apertures in, an impermeable face of a separator can be provided to allow sufficient lithium ion flux between anodic and cathodic portions of a battery.

It will be appreciated by a person skilled in the art that optimal dimensions of a separator must balance competing imperatives: maximum impedance to polysulfide migration while allowing sufficient lithium ion flux. Aside from this consideration, shape and orientation of a separator is not particularly limited, and depends in part on battery configuration. For example, in some embodiments, a separator is substantially circular in a coin-type cell, and substantially rectangular in a pouch-type cell. In some embodiments, a separator is substantially flat. However, it is not excluded that curved or other non-planar configurations may be used.

A separator may be of any suitable thickness. In order to maximize energy density of a battery, it is generally preferred that a separator is as thin and light as possible. However, a separator should be thick enough to provide sufficient mechanical robustness and to ensure suitable electrical separation of the electrodes. In certain embodiments, a separator has a thickness of from about 1 μm to about 200 μm, preferably from about 5 μm to about 100 μm, more preferably from about 10 μm to about 30 μm.

### Secondary Sulfur Battery

Described herein are secondary sulfur batteries comprising cathode compositions described above. For example, in certain embodiments, such batteries include a lithium-containing anode composition coupled to the provided cathode composition by a lithium conducting electrolyte. In some embodiments, such batteries also comprise additional components such as separators between the anode and cathode, anodic and cathodic current collectors, terminals by which a cell can be coupled to an external load, and packaging such as a flexible pouch or a rigid metal container. It is further contemplated that the present disclosure regarding secondary sulfur batteries can be adapted for use in sodium-sulfur batteries, and such batteries are also considered within the scope of certain embodiments of the present disclosure.

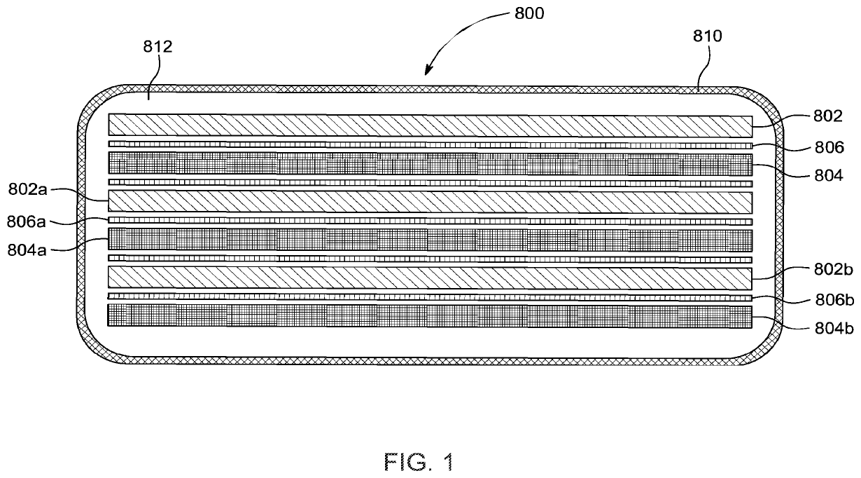

FIG. 1 illustrates a cross section of an electrochemical cell 800 in accordance with exemplary embodiments of the disclosure. Electrochemical cell 800 includes a negative electrode 802, a positive electrode 804, a separator 806 interposed between negative electrode 802 and positive electrode 804, a container 810, and a fluid electrolyte 812 in contact with negative and positive electrodes 802, 804. Such cells optionally include additional layers of electrode and separators 802a, 802b, 804a, 804b, 806a, and 806b.

Negative electrode 802 (also sometimes referred to herein as an anode) comprises a negative electrode active material that can accept cations. Non-limiting examples of negative electrode active materials for lithium-based electrochemical cells include Li metal, Li alloys such as those of Si, Sn, Bi, In, and/or Al alloys, Li₄Ti₅O₂, hard carbon, graphitic carbon, metal chalcogenides, and/or amorphous carbon. In accordance with some embodiments of the disclosure, most (e.g., greater than 90 wt %) of an anode active material can be initially included in a discharged positive electrode 804 (also sometimes referred to herein as a cathode) when electrochemical cell 800 is initially made, so that an electrode active material forms part of first electrode 802 during a first charge of electrochemical cell 800.

A technique for depositing electroactive material on a portion of negative electrode 802 is described in U.S. Patent Publication Nos. 2016/0172660 and 2016/0172661, in the name of Fischer et al., the contents of each of which are hereby incorporated herein by reference, to the extent such contents do not conflict with the present disclosure.

Positive electrode 804 (also referred to herein as cathode) comprises a cathode composition as described herein. In certain embodiments, the cathode composition comprises about 30 to about 70 wt % electroactive sulfur. In certain embodiments, a cathode comprises at least about 70% of total sulfur present in an electrochemical cell. In certain embodiments, a cathode comprises at least about 80% of total sulfur present in an electrochemical cell. In certain embodiments, a cathode comprises at least about 90% of total sulfur present in an electrochemical cell. In certain embodiments, a cathode comprises at least about 95% of total sulfur present in an electrochemical cell. In certain embodiments, a cathode comprises at least about 99% of total sulfur present in an electrochemical cell. In certain embodiments, a cathode comprises essentially all of the total sulfur present in an electrochemical cell.

Negative electrode 802 and positive electrode 804 can further include one or more electrically conductive additives as described herein. In accordance with some embodiments of the disclosure, negative electrode 802 and/or positive electrode 804 further include one or more polymer binders as described herein.

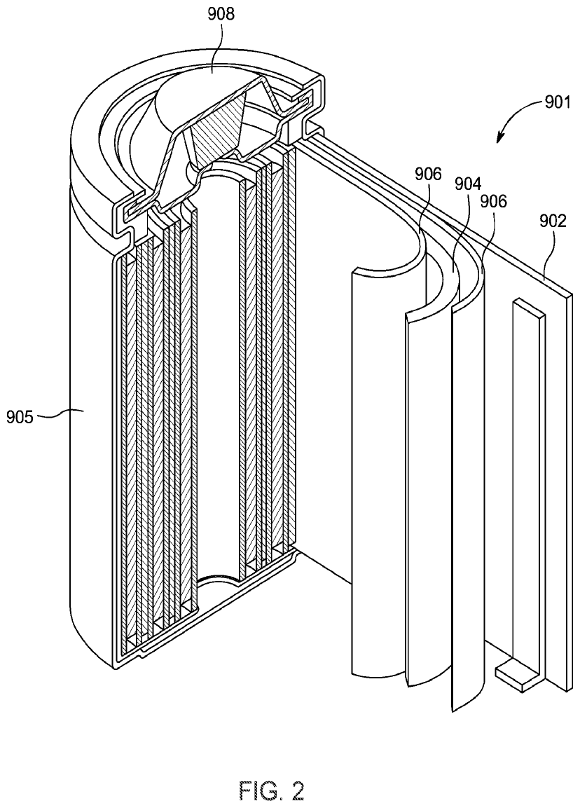

FIG. 2 illustrates an example of a battery according to various embodiments described herein. A cylindrical battery is shown here for illustration purposes, but other types of arrangements, including prismatic or pouch (laminate-type) batteries, may also be used as desired. Example Li battery 901 includes a negative anode 902, a positive cathode 904, a separator 906 interposed between the anode 902 and the cathode 904, an electrolyte (not shown) impregnating the separator 906, a battery case 905, and a sealing member 908 sealing the battery case 905. It will be appreciated that example battery 901 may simultaneously embody multiple aspects of the present disclosure in various designs.

A secondary sulfur battery of the present disclosure comprises a lithium anode, a porous sulfur-based cathode, and an electrolyte permitting lithium ion transport between anode and cathode. In certain embodiments, described herein, an anodic portion of a battery comprises an anode and a portion of electrolyte with which it is in contact. Similarly, in certain embodiments, described herein, a cathodic portion of a battery comprises a cathode and a portion of electrolyte with which it is in contact. In certain embodiments, a battery comprises a lithium ion-permeable separator, which defines a boundary between an anodic portion and a cathodic portion. In certain embodiments, a battery comprises a case, which encloses both anodic and cathodic portions. In certain embodiments, a battery case comprises an electrically conductive anodic-end cover in electrical communication with an anode, and an electrically conductive cathodic-end cover in electrical communication with a cathode to facilitate charging and discharging via an external circuit.

In certain embodiments, a secondary sulfur battery of the present disclosure is defined in terms of its ratio of electrolyte to electroactive sulfur. Electrolyte volume and the ratio (vol/wt) of electrolyte to sulfur in a cathode correlate to energy density of a sulfur battery. Electrolyte may be distributed among different volumes within a cell, for example electrolyte may be contained in porosity of the cathode, in the separator, and in contact with the anode or within an anodic solid electrolyte interphase. Electrolyte may also be contained in other spaces within a battery where it is not in direct contact with the anodic or cathodic active materials—for example electrolyte may be stranded in an annular volume at the edges of a coin cell. In certain embodiments, the present invention provides batteries where all or most of the electrolyte is contained within the cathode. Preferably, substantially all of the electrolyte is contained within the cathode and only a minimal amount of electrolyte that is necessary to wet the separator and the anode surface or SEI is outside of the cathode. Electrolyte contained within the cathode is referred to as “contained electrolyte” and its volume V_{CE} can be estimated as theoretical pore volume, or porosity multiplied by the geometric volume of a cathode film:

$${V}_{CE} = P \% \times Area \times {thickness}_{cathode}$$

In certain embodiments, a provided secondary sulfur battery is characterized in that at least 50% of the total electrolyte inventory (Vₜₒₜ) is contained in the cathode (e.g. V_{CE}/Vₜₒₜ>0.5). In certain embodiments, a provided secondary sulfur battery is characterized in that at least 50% of the total electrolyte inventory (Vₜₒₜ) is contained in the cathode (e.g. V_{CE}/Vₜₒₜ>0.8). In certain embodiments, a secondary sulfur battery has at least 60%, at least 65%, or at least 70% of the electrolyte contained in the cathode porosity. In certain embodiments, a secondary sulfur battery has at least 80%, at least 85%, or at least 90%, of the electrolyte contained in the cathode porosity. In certain embodiments, a secondary sulfur battery has at least 92%, at least 94%, at least 95%, at least 96%, or at least 97% of the electrolyte contained in the cathode.

The ratio of total electrolyte-to-sulfur (E/S) is another parameter that influences the energy density of a battery. The E/S ratio is calculated based on the total volume of electrolyte Vₜₒₜ and the mass of electroactive sulfur (m_{sulfur}):

$$\frac{E}{S} = \frac{{V}_{tot}}{{m}_{sulfur}}$$

In certain embodiments, a secondary sulfur battery has an electrolyte-to-sulfur ratio equal to or less than about 6 microliters of electrolyte per milligram of electroactive sulfur. In certain embodiments, a secondary sulfur battery has an electrolyte-to-sulfur ratio equal to or less than about 5 microliters of electrolyte per milligram of electroactive sulfur. In certain embodiments, a secondary sulfur battery has an electrolyte-to-sulfur ratio equal to or less than about 4.5 microliters of electrolyte per milligram of electroactive sulfur. In certain embodiments, a secondary sulfur battery has an electrolyte-to-sulfur ratio equal to or less than about 3.5 microliters of electrolyte per milligram of electroactive sulfur or less than about 3.0 microliters of electrolyte per milligram of electroactive sulfur. In certain embodiments, a secondary sulfur battery has an electrolyte-to-sulfur ratio equal to or less than about 3.5 microliters of electrolyte per milligram of electroactive sulfur. In certain embodiments, a secondary sulfur battery has an electrolyte-to-sulfur ratio equal to or less than about 3 microliters of electrolyte per milligram of electroactive sulfur. In certain embodiments, a secondary sulfur battery has an electrolyte-to-sulfur ratio between about 1.8 and about 3.5 μL/mg S. In certain embodiments, a secondary sulfur battery has an electrolyte-to-sulfur ratio between about 1.8 and about 2.5 μL/mg S. In certain embodiments, a secondary sulfur battery has an electrolyte-to-sulfur ratio between about 1.0 and about 2.0 μL/mg S. In certain embodiments, a secondary sulfur battery has an electrolyte-to-sulfur ratio between about 1.5 and about 2.0 μL/mg S.

Additives disclosed herein do not necessarily need to be used as electrolyte solvent replacements. In some embodiments, an additive is used with an ether-based solvent in an electrolyte. In some embodiments, an additive is present in an electrolyte at a concentration in a range of from 3 mM to 0.5 M, for example from 10 mM to 0.5 M, from 3 mM to 0.2 M, from 10 mM to 0.2 M, from 50 mM to 0.5 M, from 50 mM to 0.2 M.

In some embodiments, a lithium-sulfur battery of the present disclosure comprises a lithium anode, a sulfur-based cathode, and an electrolyte permitting ion transport between anode and cathode. In certain embodiments, described herein, an anodic portion of a battery comprises an anode and a portion of electrolyte with which it is in contact. Similarly, in certain embodiments, described herein, a cathodic portion of a battery comprises a cathode and a portion of electrolyte with which it is in contact. In certain embodiments, a battery comprises a lithium ion-permeable separator, which defines a boundary between an anodic portion and a cathodic portion. In certain embodiments, a battery comprises a case, which encloses both anodic and cathodic portions. In certain embodiments, a battery case comprises an electrically conductive anodic-end cover in electrical communication with an anode, and an electrically conductive cathodic-end cover in electrical communication with a cathode to facilitate charging and discharging via an external circuit.

### EXEMPLIFICATION

The following examples embody certain methods of the present disclosure and demonstrate fabrication of porous cathode composites, and secondary sulfur batteries comprising the same, according to certain embodiments described herein. Moreover, the following examples are included to demonstrate principles of disclosed compositions and methods and are not intended as limiting.

### Example 1: Preparation and Characteristics of Electrochemical Cells with Electrolyte Additives

The present example demonstrates various characteristics of exemplary electrochemical cells according to embodiments disclosed herein. An exemplary electrochemical cell includes a sulfur-containing cathode, a lithium-containing anode, an electrolyte, and a separator.

An electroactive material for use in the sulfur-containing cathode was prepared by heat melt diffusion of sulfur and carbon black for 3.5 hours at 130° C., followed by 16 hours at 170° C. Next, the active material was cooled, milled, and processed using a 60 μm sieve. The cathode was prepared using a high speed bladeless mixer in which solvent, active materials, PVDF binder, and carbon black were mixed. The mixture was cast onto a carbon-coated aluminum current collector at a target loading within a range of about 5-6 mg Sulfur/cm². The cast cathode was dried at 60° C. in a vacuum with gas sweep.

An electrolyte was prepared by mixing 1,2-dimethoxyethane, 1,3 dioxolane, a lithium salt, LiNO₃, and one of: 3H-1,2-benzodithiol-3-one, phenylacetyl disulfide, tetramethylthiourea, thioacetamide, thiourea, trithiocyanuric acid, vinylene trithiocarbonate, zinc dimethyldithiocarbamate, and dimethyl trithiocarbonate.

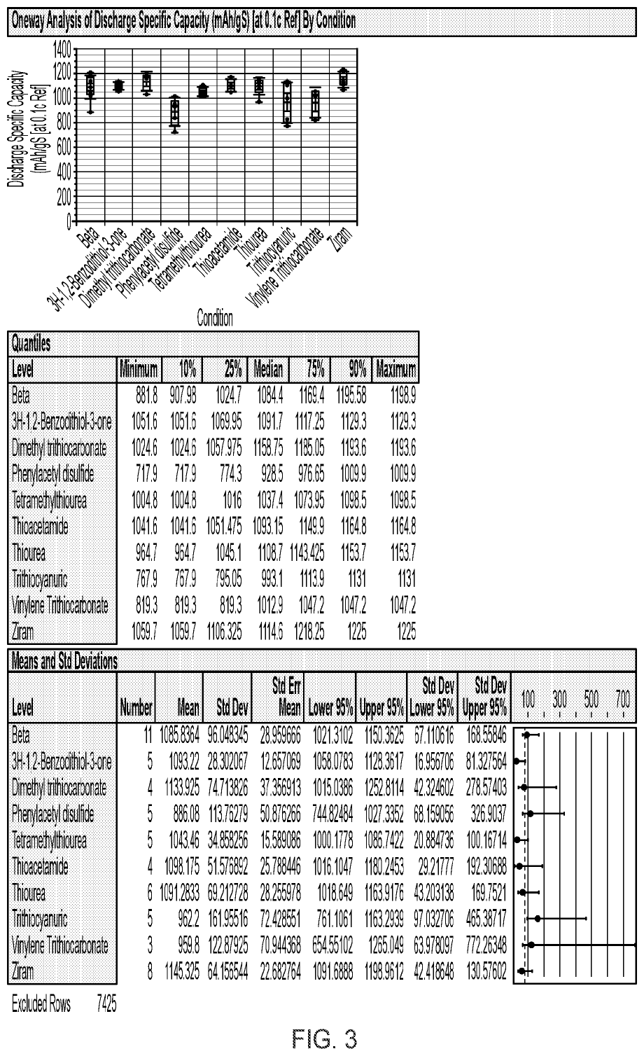

These electrochemical cells which include electrolytes comprising additives were tested in comparison to control electrochemical cells which were prepared similarly but differ in electrolyte composition. The electrolyte composition for the control cells includes a mixture consisting of 1,2-dimethoxyethane, 1,3 dioxolane, a lithium salt, and LiNO₃. Control cells are referred to as “Beta” throughout FIGS. 3-11 and their corresponding data.

Discharge Specific Capacity at 0.1 c: FIG. 3 presents data for discharge specific capacity at 0.1 c for electrochemical cells that were tested. Without wishing to be bound by any particular theory, discharge specific capacity at 0.1 c is useful in understanding the utilization and/or energy of the cell. As seen in FIG. 3, a cell comprising an electrolyte with ziram provided the best performance relative to other additives in terms of discharge specific capacity at 0.1 c.

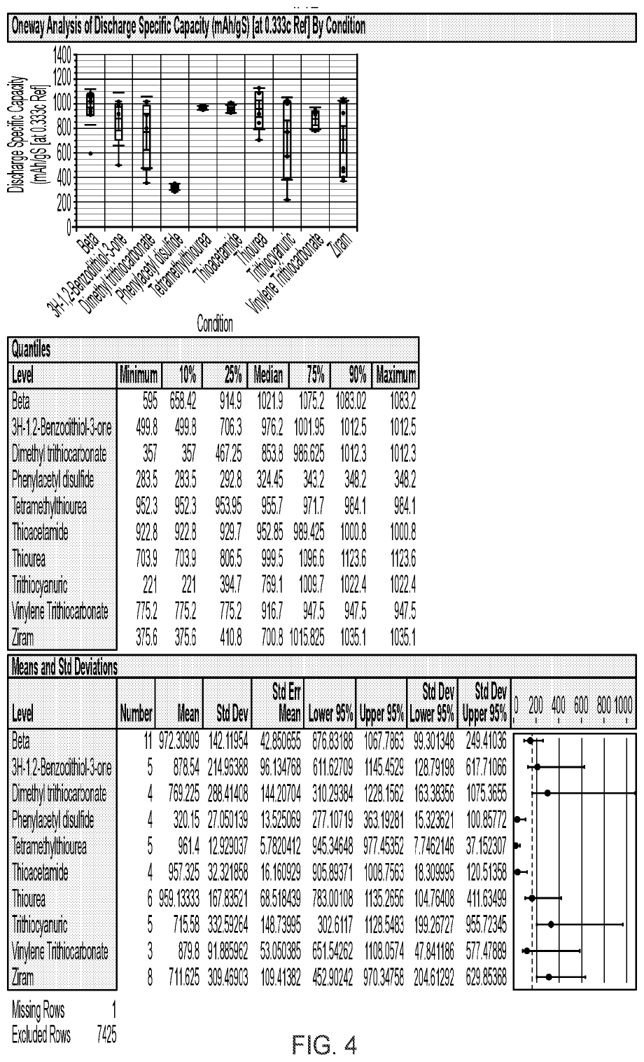

Discharge Specific Capacity at 0.333 c: FIG. 4 presents data for discharge specific capacity at 0.333c for electrochemical cells that were tested. Without wishing to be bound by any particular theory, discharge specific capacity at 0.333 c is useful in understanding the rate capability and/or power capability of the cell. As seen in FIG. 4, cells comprising an electrolyte comprising one of tetramethyl. thiourea, thioacetamide, thiourea, and ziram performed best relative to other cells. One common feature across each compound is that each comprises an NCS moiety. Additionally, trithiocyanuric, which has an aromatic core comprising an NCS moiety, performed similarly to tetramethyl, thiourea, thioacetamide, thiourea, and ziram. Without wishing to be bound by any particular theory, increased performance for each of the aforementioned additives could be attributed to each compound having an NCS moiety and a thiocarbonyl group.

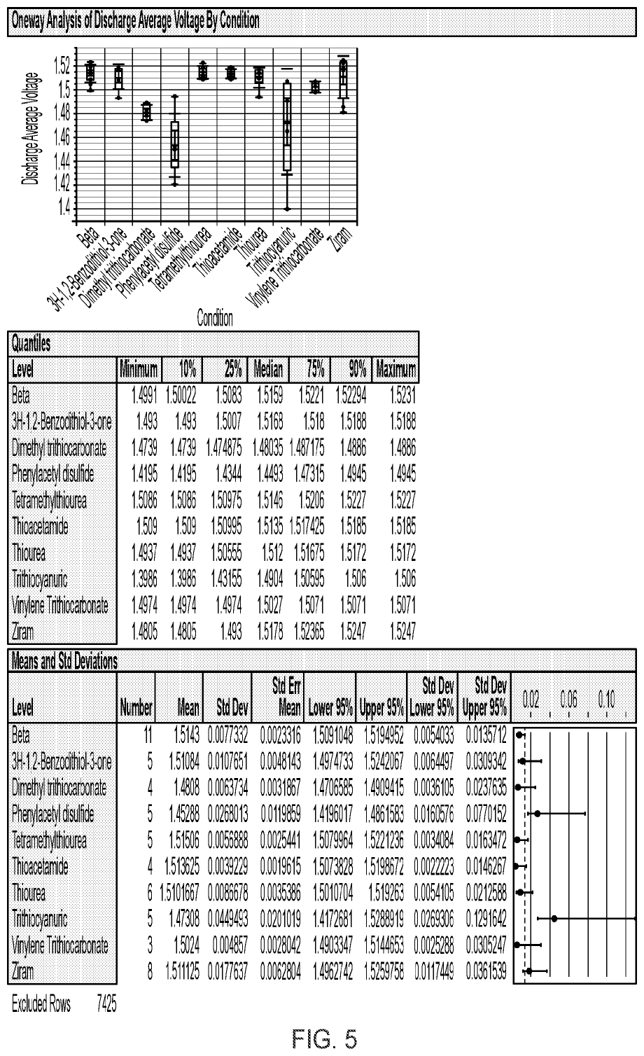

Average Discharge Voltage: FIG. 5 presents data for average discharge voltage average discharge voltage, wherein the discharge voltage is a capacity normalized voltage. Without being bound by any theory, improved cell performance is typically associated with higher average discharge voltage, which can be indicative of lower resistance. As seen in FIG. 5, tetramethylthiourea, thioacetamide, thiourea, vinylene trithiocarbonate, and ziram demonstrated increased performance relative to other additives.

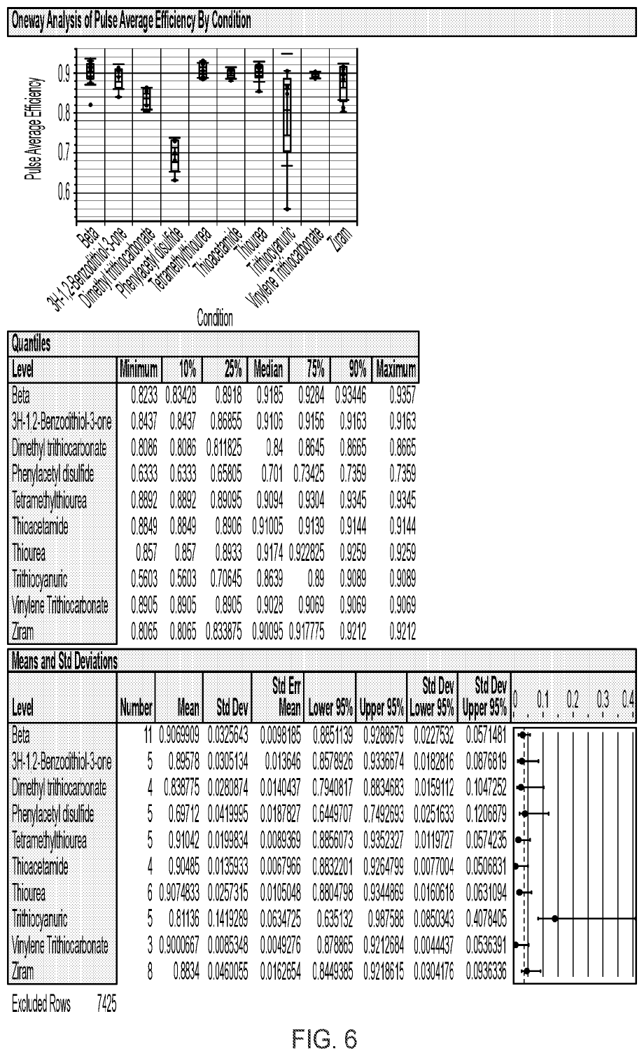

Pulse Average Efficiency: FIG. 6 presents pulse average efficiency, which is determined as the average of each of the pulse efficiencies across the discharge, wherein pulse efficiency was determined as the integrated area of C/10 voltage divided by the integrated area of 1 C voltage during the 5 second pulses. As seen in FIG. 6, tetramethylthiourea, thioacetamide, and thiourea, demonstrated increased performance relative to other additives.

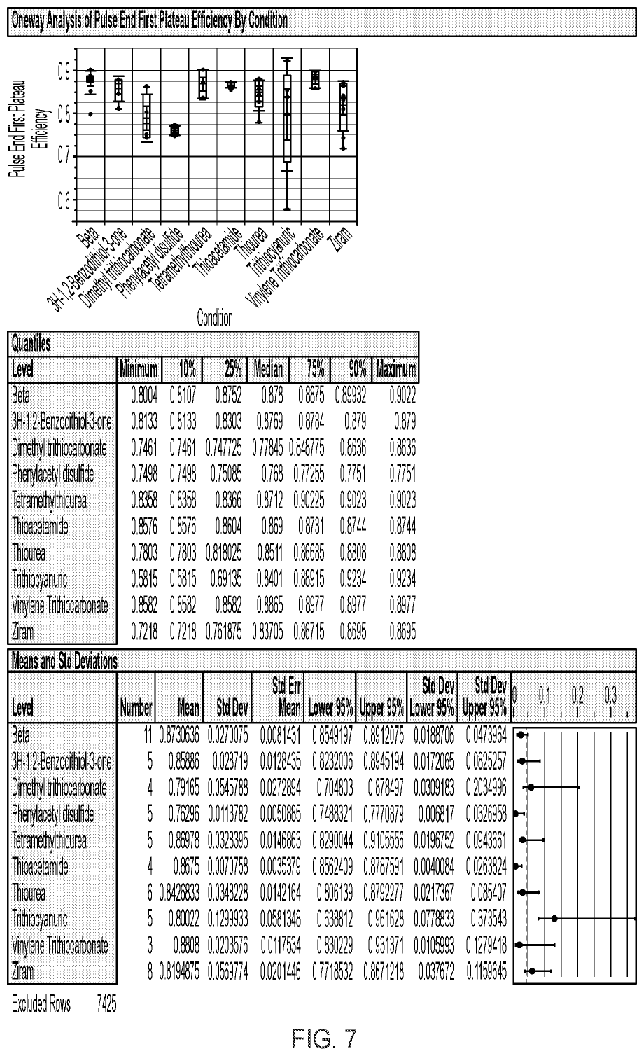

Pulse End First Plateau Efficiency: FIG. 7 presents data for pulse end first plateau efficiency, which was determined as the pulse efficiency for the first pulse that is both >300 mAh/gS and 25% depth of discharge. As seen in FIG. 7, tetramethylthiourea, thioacetamide, and thiourea, demonstrated increased performance relative to other additives.

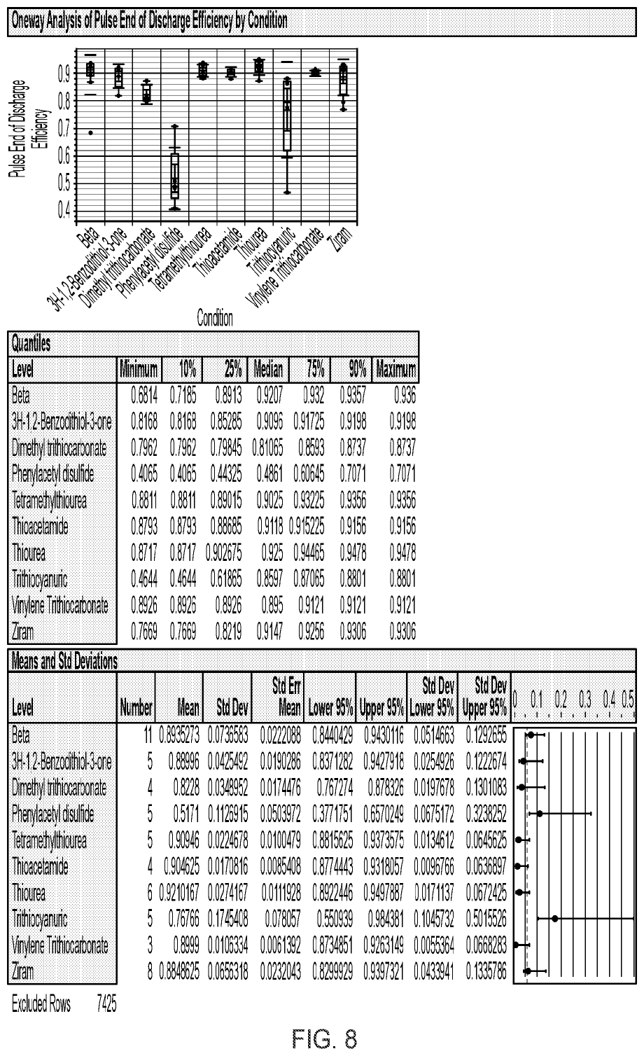

Pulse End of Discharge Efficiency: FIG. 8 presents data for pulse end of discharge efficiency, which was determined as the pulse efficiency averaged across pulses that are both >600 mAh/gS and 75% depth of discharge). As seen in FIG. 8, tetramethylthiourea, thioacetamide, and thiourea, demonstrated increased performance relative to other additives.

Without being bound by any particular theory, it was important to observe to the strong performance of tetramethylthiourea, thioacetamide, and thiourea, across the testing demonstrated in FIGS. 6-8 as this was indicative of consistent cell performance across cell discharge.

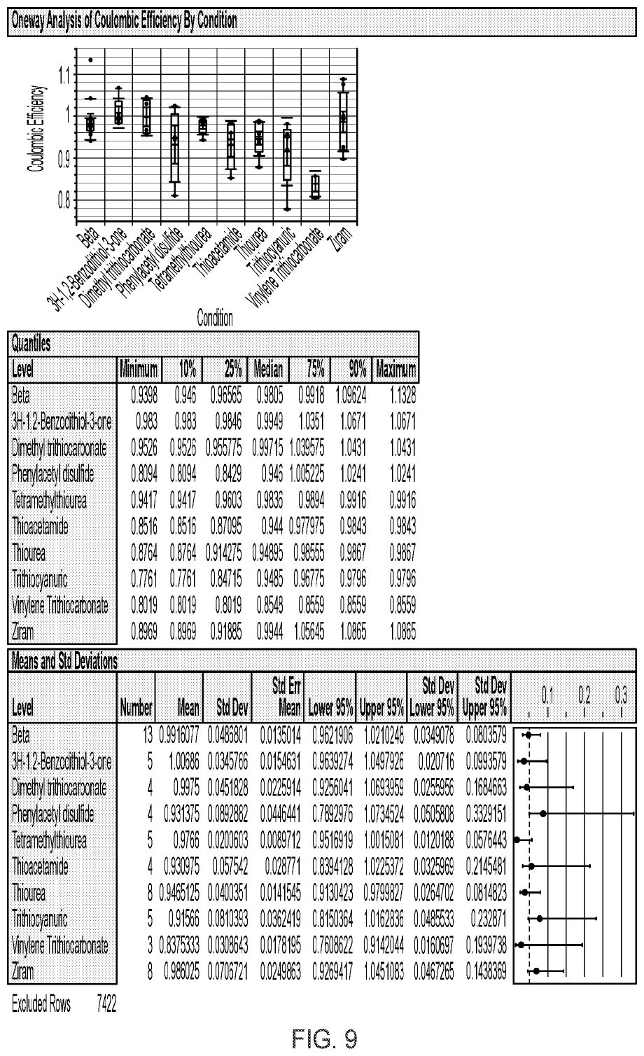

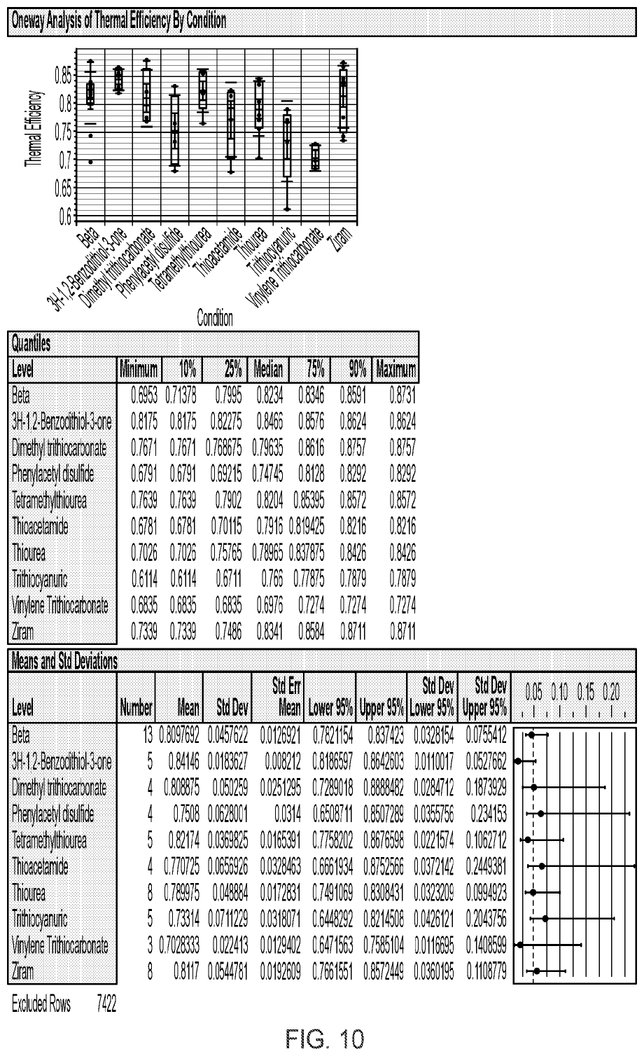

Coulombic Efficiency: FIG. 9 presents data for coulombic efficiency which was determined as the ratio of discharge capacity divided by the charge capacity. Without being bound by any theory, coulombic efficiency is indicative of a cell's maximum limit of its cycle life performance. As presented in FIG. 10, each additive performed similarly relative to each other.

Thermal Efficiency: FIG. 10 presents data for thermal efficiency which was determined as the ratio of the discharge energy divided by the charge energy over the course of one full cycle of the electrochemical cell. Without being bound by any theory, an increased value of thermal efficiency indicates increased management of heat within the cell. As presented in FIG. 10, each additive performed similarly relative to each other.

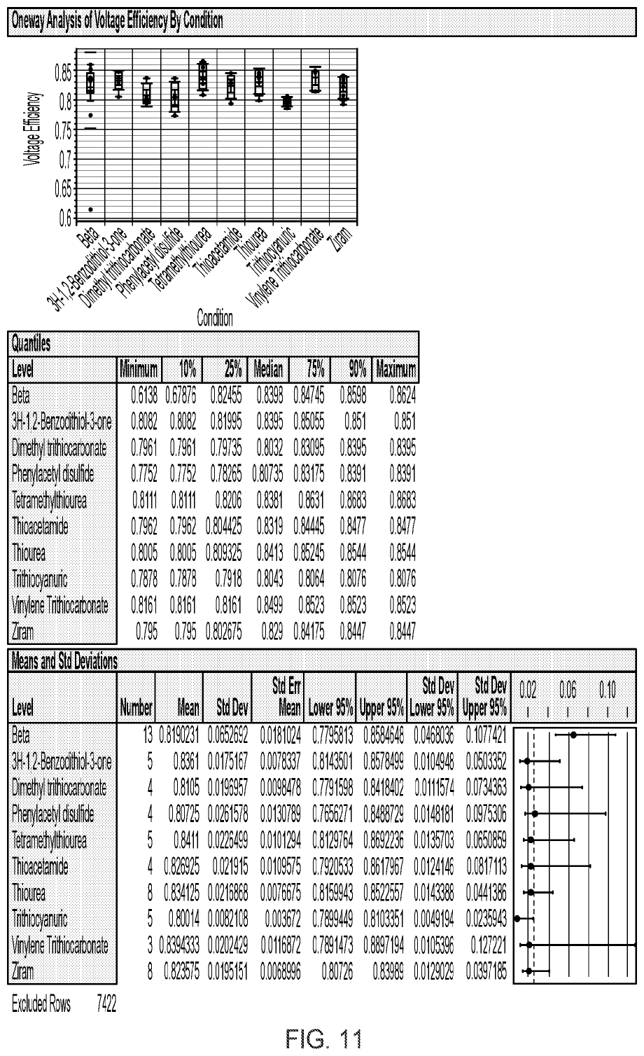

Voltage Efficiency. FIG. 11 presents data for voltage efficiency, which was determined as the ratio of the average discharge voltage divided by the average charge voltage over the course of one full cycle of the electrochemical cell. As presented in FIG. 11, each additive performed similarly relative to each other.

### Example 2: Preparation and Characteristics of Electrochemical Cells with Functional Binders

An electroactive material for use in the sulfur-containing cathode can be prepared by heat melt diffusion of sulfur and carbon black for 3.5 hours at 130° C., followed by 16 hours at 170° C. Next, the active material can be cooled, milled, and processed using a 60 m sieve. The cathode can be prepared using a high speed bladeless mixer in which solvent, active material, a binder comprising a thiocarbonyl functional group, and carbon black are mixed. The mixture can be cast onto a carbon-coated aluminum current collector at a target loading within a range of about 5-6 mg sulfur/cm². The cast cathode can then be dried at 60° C. in a vacuum with gas sweep.

### Example 3: Electrochemical Characteristics of Cells Comprising Electrolytes with Disclosed Additives

To evaluate the effect of additives of the present disclosure on the performance of lithium-sulfur secondary batteries, coin cells can be assembled. A cathode material can be prepared as described in Example 1. For example, a mixture of an active material (e.g., 75 wt % of active material comprising a mixture of ˜80 wt % elemental sulfur and ˜20 wt % polyaniline), conductive carbon additive (e.g., 14 wt % C₆₅®), and binder (e.g., 11 wt % PVDF) can be prepared. These components are combined in a minimal amount of solvent (e.g., NMP) and mixed to form a homogenous slurry. The resulting slurry is applied to carbon coated Al foil, and dried overnight prior to use. Disks are punched from the cathode film (e.g., diameters of 1.27 cm). The final sulfur loading on each cathode can be about 3 g/cm².

CR2032 coin cells may be assembled using cathode punches in combination with the following components:
Anode, e.g., a 0.2 mm thick Li-metal disc with a 9/16″ diameter
   Separator, e.g., Celgard-0325

### Electrolyte:

Electrolyte (e.g., 1 M LiTFSI and 0.2 M LiNO₃ in a 1:1 mixture of DME:DOL by volume) is added to each coin cell in a sufficient amount to provide cells with the desired E:S ratios. For example, for an E:S of ˜3, 13 μL of electrolyte may be used for each coin cell.
   Electrochemical testing may be conducted at room temperature using a Maccor 4000 battery tester. The cycling protocol may include the following steps:
1. An initial rest period of 3 hours
2. An initial discharge at a rate of C/20—labeled cycle 0
3. A charge/discharge cycle at a rate of C/20—labeled cycle 1
4. A charge/discharge cycle at a rate of C/10—labeled cycle 2
5. 9 charge/discharge cycles at a rate of C/3
6. A charge/discharge cycle at a rate of C/10
7. 30 repeats of steps 4 & 5 (300 total cycles)
   A 10 minute rest period may be applied after the conclusion of each charge & discharge cycle.
A suitable upper voltage cutoff bound is 2.8 V vs. Li⁺/Li
A suitable lower voltage cutoff bound is 1.7 V vs. Li⁺/Li
   The discharge capacity of an electrochemical cell is measured using the cycling protocol described above.

### Example 4: Preparation of Thiocarbonyl During Discharge

The present example demonstrates the preparation of a thiocarbonyl additive during battery cycling. A slurry can be prepared by mixing 3H-1,2-benzodithiol-3-one and phenylacetyl disulfide and can be included with a lithium polythioacrlyate binder in an electrochemical cell (e.g., wherein the polythioacrylate binder is included in a proportion of no more than 20 wt %). Without being bound by any theory, during operation of an electrochemical cell which includes the aforementioned components, disulfide bonds of the slurry components are reduced at the anode or cathode of the electrochemical cell at the start of discharge and a polyacrylic acid thiocarboxylate analogue is produced, the thiocarboxylate analogue having a resonance form which includes a thiocarbonyl.

### EXEMPLARY ENUMERATED EMBODIMENTS

The following numbered embodiments, while non-limiting, are exemplary of certain aspects of the present disclosure:
1. A binder for a sulfur cathode comprising a thiocarbonyl functional group.
2. An additive for an electrolyte in a lithium sulfur battery comprising a thiocarbonyl functional group.
3. An electrolyte for a lithium sulfur battery comprising a thiocarbonyl functional group.
4. The binder, additive, or electrolyte of any one of the previous embodiments, comprising thiocarbonyl functional groups of formula X_{y}C═S, where each X is independently selected from oxygen, nitrogen, sulfur, or carbon, and y is 1 or 2; wherein one X can be taken together with the other X and intervening atoms to form a ring; and when y is 1, X is connected to the thiocarbonyl carbon via a double bond.
5. The binder, additive, or electrolyte of embodiment 4, wherein y is 2.
6. The binder, additive, or electrolyte of embodiment 4 or 5, wherein each X is independently selected from nitrogen, sulfur, or carbon.
7. The binder, additive, or electrolyte of embodiment 4 or 5, wherein each X is independently selected from sulfur or carbon.
8. The binder, additive, or electrolyte of embodiment 4, wherein y is 1.
9. The binder, additive, or electrolyte of embodiment 8, comprising isothiocyanate functional groups.
10. The binder, additive, or electrolyte of any one of the previous embodiments, wherein the binder, additive, or electrolyte is uncycled.
11. A compound of Formula I′:

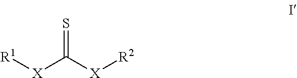

wherein R¹ and R² are each independently hydrogen or an optionally substituted group selected from C₁₋₁₅ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur;
   or R¹ and R² are taken together with intervening atoms to form an optionally substituted ring;
      each X is absent or independently selected from O, S, NR^{z}, and CR³R⁴;
      each R¹ is independently hydrogen or optionally substituted CL-12 aliphatic;
each R³ and R⁴ is independently hydrogen, halogen, —CN, —NO₂, —N(R)₂, —OR, —SR, or an optionally substituted group selected from C₁₋₁₂ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
each R is independently hydrogen, optionally substituted C₁₋₆ aliphatic, optionally substituted 3- to 7-membered saturated or partially unsaturated carbocyclyl, or optionally substituted 3- to 7-membered saturated or partially unsaturated monocyclic heterocyclyl having 1-3 heteroatoms independently selected from nitrogen, oxygen, and sulfur, or two R when attached to the same nitrogen atom are taken together form an optionally substituted 3- to 7-membered saturated or partially unsaturated monocyclic heterocyclyl having 0-2 additional heteroatoms independently selected from nitrogen, oxygen, and sulfur.
12. A compound of Formula I:

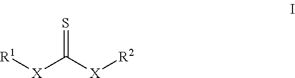

wherein R¹ and R² are each independently an optionally substituted group selected from C₁₋₁₅ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur;
   or R¹ and R² are taken together with intervening atoms to form an optionally substituted ring;
      each X is absent or independently selected from O, S, NR^{z}, and CR³R⁴;
      each R^{z} is independently hydrogen or optionally substituted C₁₋₁₂ aliphatic;
each R³ and R⁴ is independently hydrogen, halogen, —CN, —NO₂, —N(R)₂, —OR, —SR, or an optionally substituted group selected from C₁₋₁₂ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
each R is independently hydrogen, optionally substituted C₁₋₆ aliphatic, optionally substituted 3- to 7-membered saturated or partially unsaturated carbocyclyl, or optionally substituted 3- to 7-membered saturated or partially unsaturated monocyclic heterocyclyl having 1-3 heteroatoms independently selected from nitrogen, oxygen, and sulfur, or two R when attached to the same nitrogen atom are taken together form an optionally substituted 3- to 7-membered saturated or partially unsaturated monocyclic heterocyclyl having 0-2 additional heteroatoms independently selected from nitrogen, oxygen, and sulfur.
13. The compound of embodiment 11 or 12, wherein R¹ and R² are taken together with intervening atoms to form an optionally substituted ring selected from 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.
14 The compound of embodiment 13, wherein R¹ and R² are taken together with intervening atoms to form an optionally substituted 3- to 7-membered saturated or partially unsaturated monocyclic heterocyclyl having 1-3 heteroatoms independently selected from nitrogen, oxygen, and sulfur.
15. The compound of embodiment 14, wherein R¹ and R² are taken together with intervening atoms to form an optionally substituted 5-membered saturated or partially unsaturated monocyclic heterocyclyl having 2 sulfur heteroatoms.
16. The compound of embodiment 13, wherein R¹ and R² are taken together with intervening atoms to form an optionally substituted 5- to 6-membered monocyclic heteroaryl having 1-4 heteroatoms independently selected from nitrogen, oxygen, and sulfur.
17. The compound of embodiment 13, wherein R¹ and R² are taken together with intervening atoms to form Ring A as in Formula II.

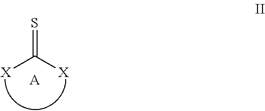

Ring A is an optionally substituted ring selected from 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.
18. The compound of embodiment 17, wherein Ring A is optionally substituted 5-membered saturated or partially unsaturated monocyclic heterocyclyl having 2 sulfur heteroatoms.
19. The compound of embodiment 17, wherein Ring A is optionally substituted 5- to 6-membered monocyclic heteroaryl having 1-4 heteroatoms independently selected from nitrogen, oxygen, and sulfur.
20. The compound of any one of embodiments 11-19, wherein both X are absent.
21. The compound of any one of embodiments 11-19, wherein both X are S.
22. The compound of any one of embodiments 11-19, wherein both X are NR.
23. The compound of embodiment 11 or 12, wherein the compound is trithiocyanuric acid.
24. A binder, additive, or electrolyte comprising a compound of any one of embodiments 11-23.
25. A binder, additive, or electrolyte comprising a compound selected from the group consisting of 3H-1,2-benzodithiol-3-one, phenylacetyl disulfide, tetramethylthiourea, thioacetamide, thiourea, trithiocyanuric acid, vinylene trithiocarbonate, zinc dimethyldithiocarbamate, and dimethyl trithiocarbonate.
26. A binder, additive, or electrolyte comprising a compound selected from the group consisting of

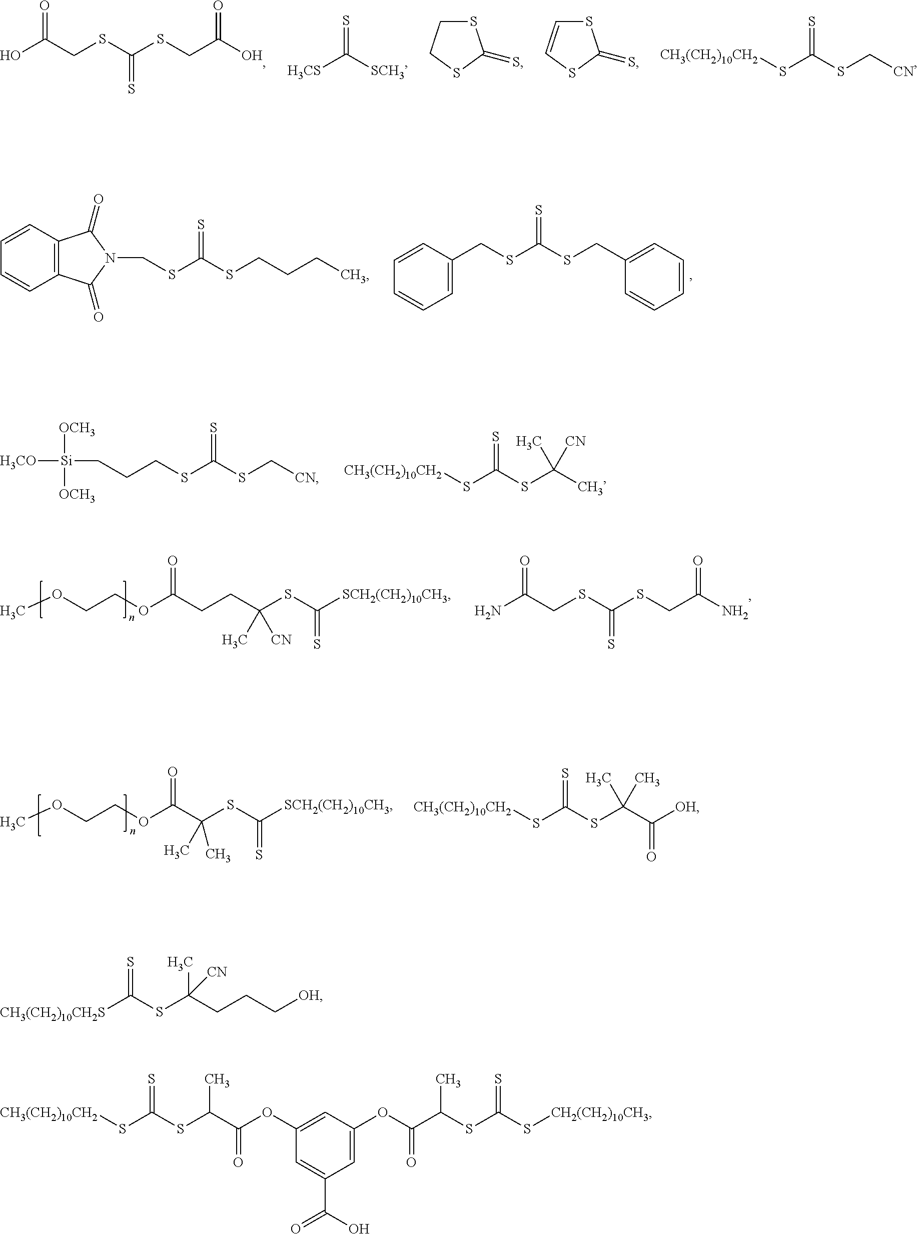

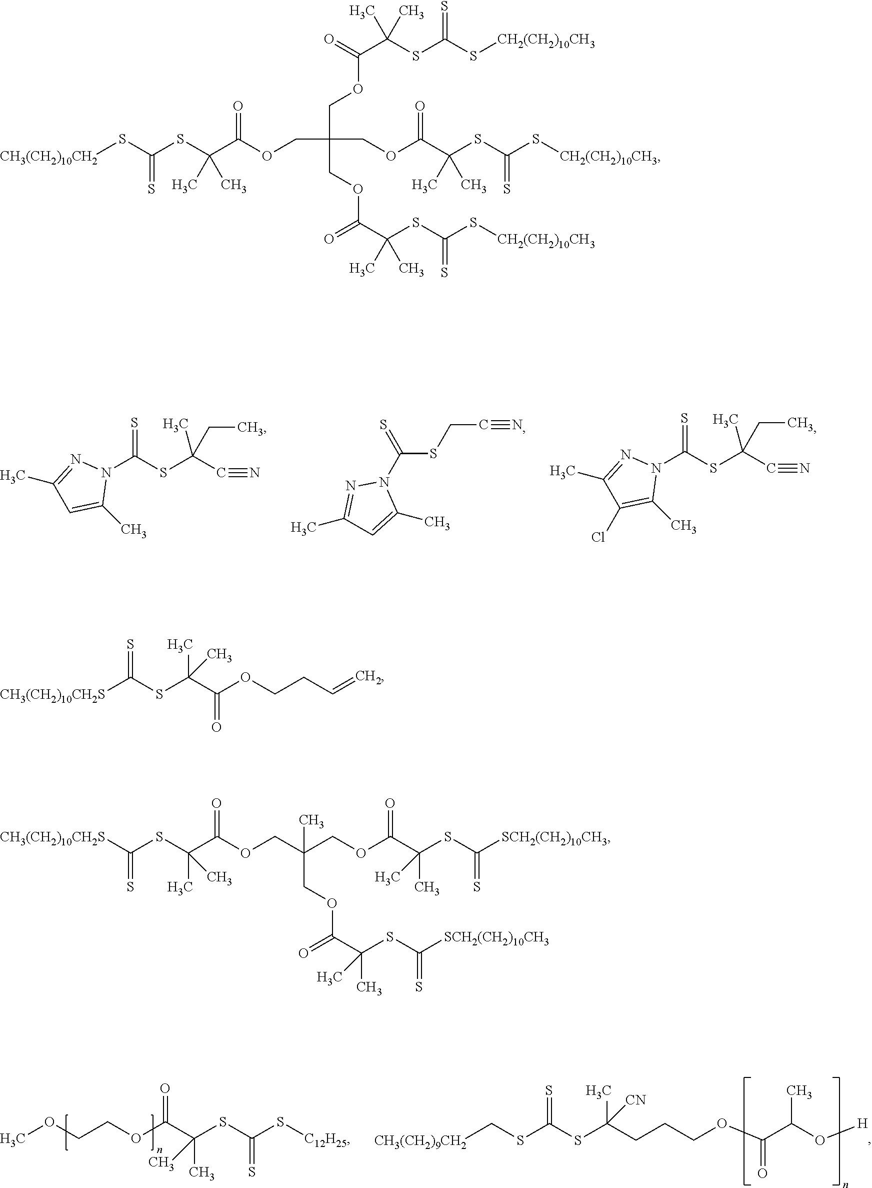

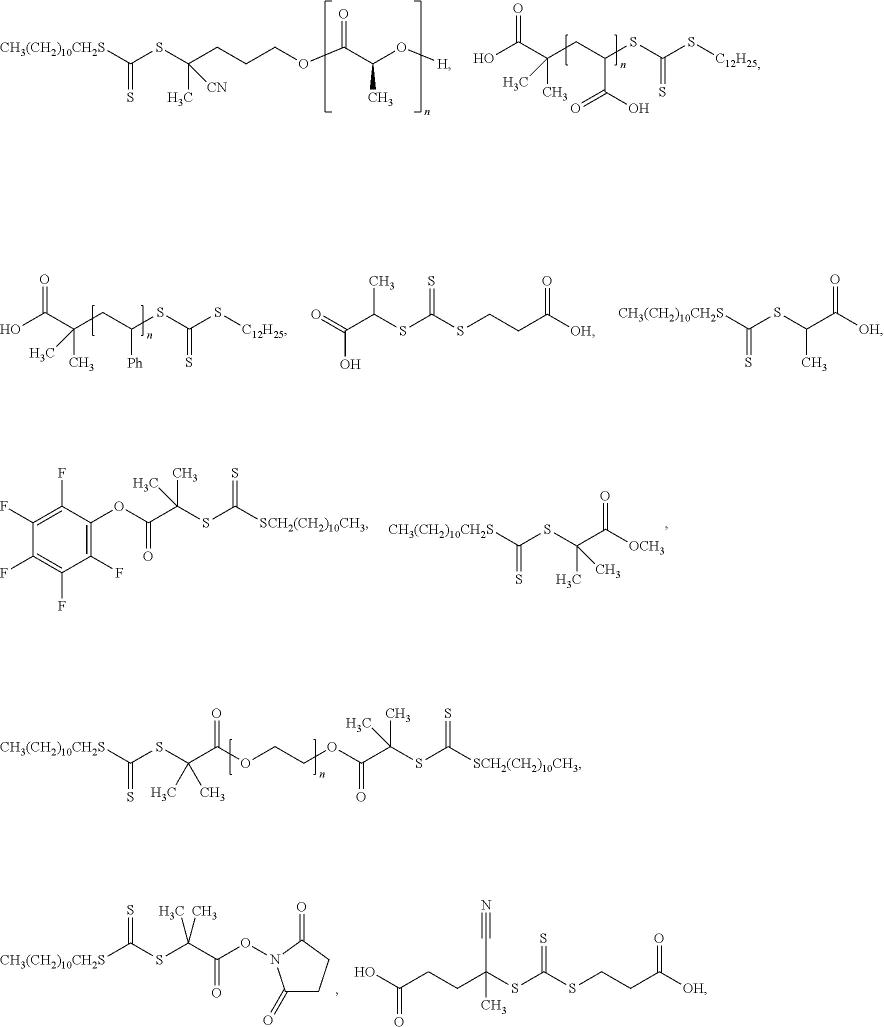

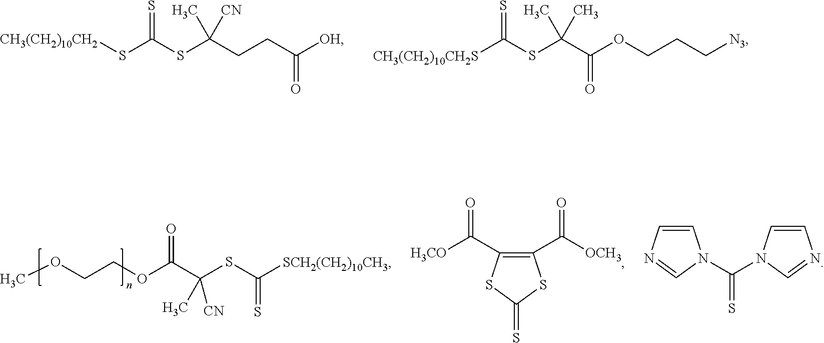

27. An electrolyte composition comprising a compound selected from the group consisting of 3H-1,2-benzodithiol-3-one, phenylacetyl disulfide, tetramethylthiourea, thioacetamide, thiourea, trithiocyanuric acid, vinylene trithiocarbonate, zinc dimethyldithiocarbamate, and dimethyl trithiocarbonate.
28. An electrolyte composition comprising a compound selected from the group consisting of

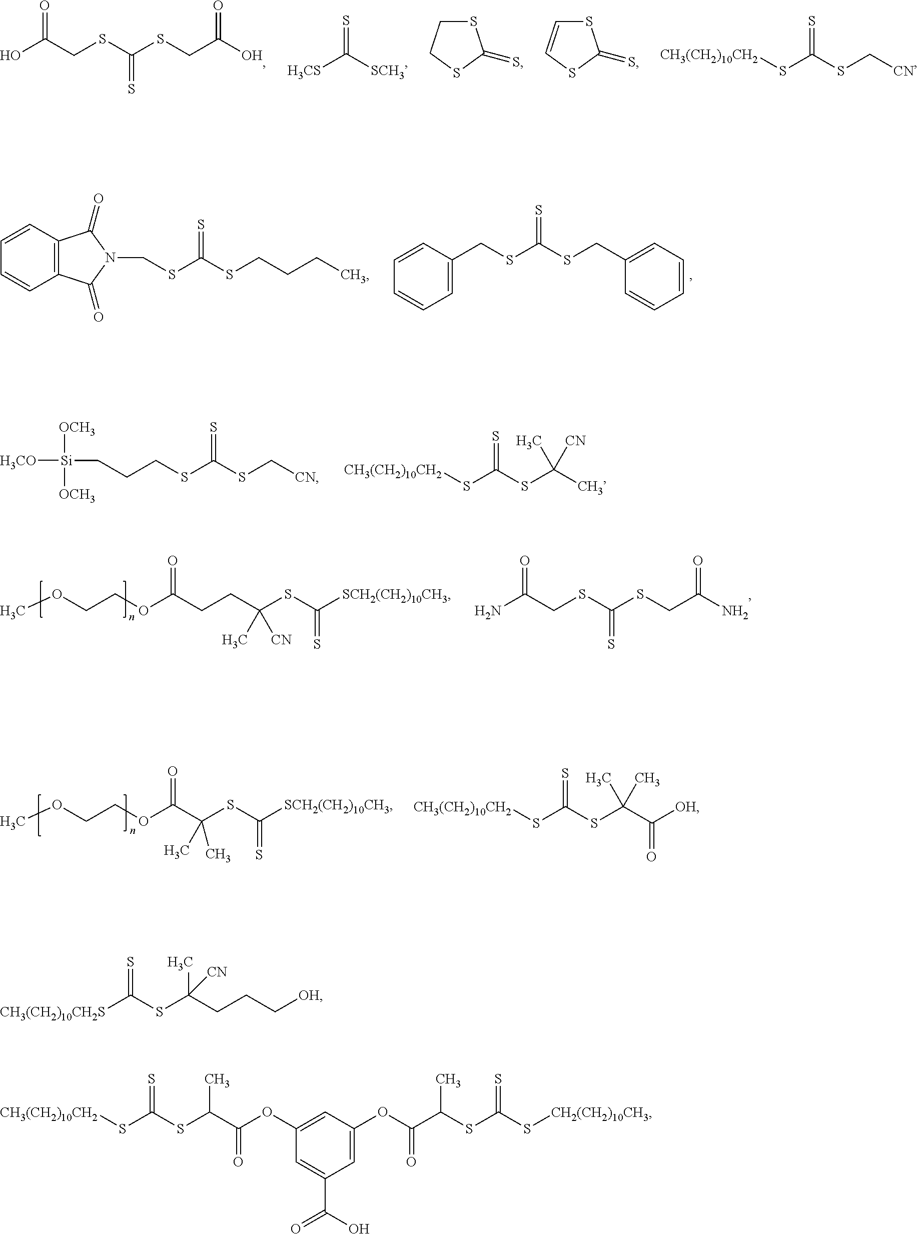

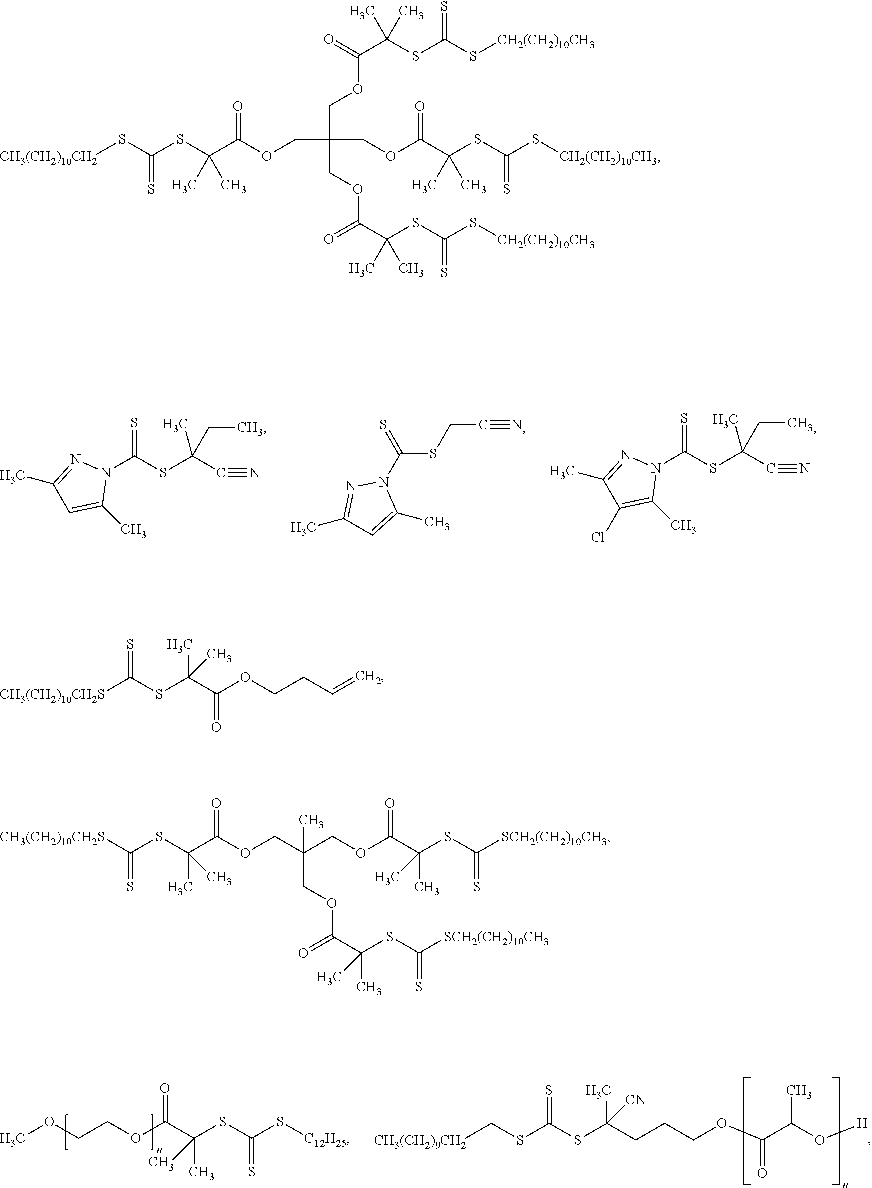

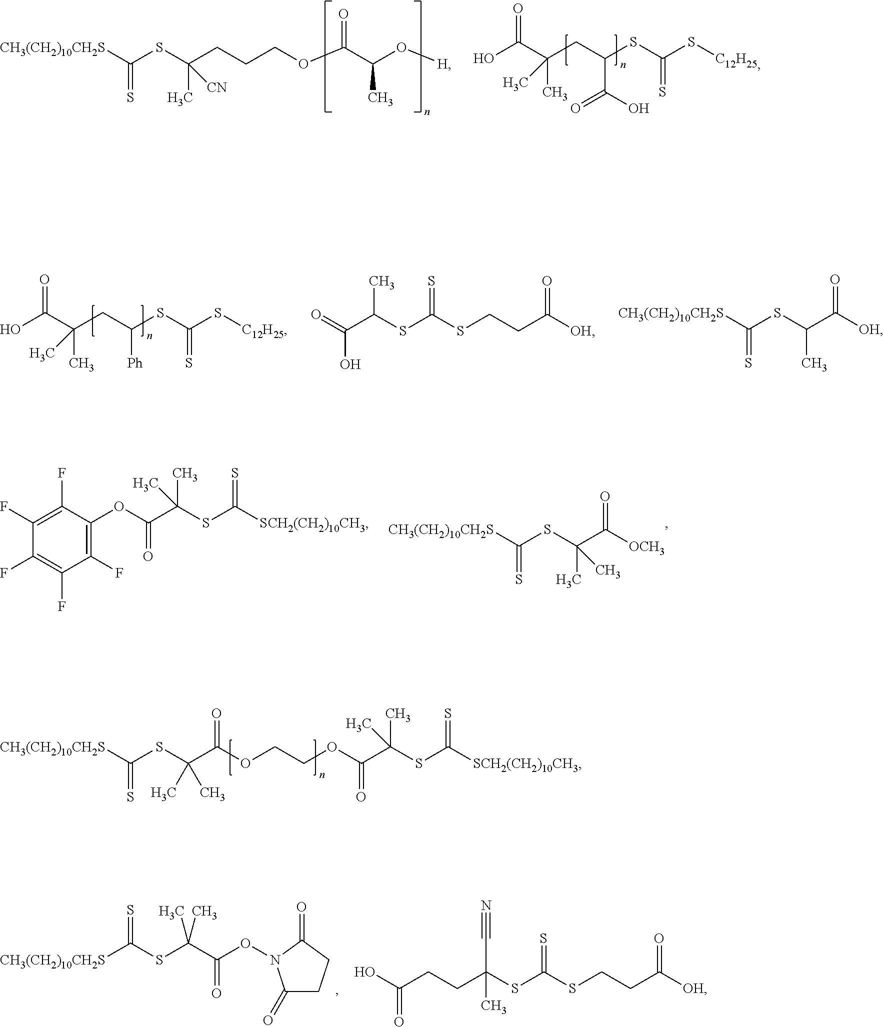

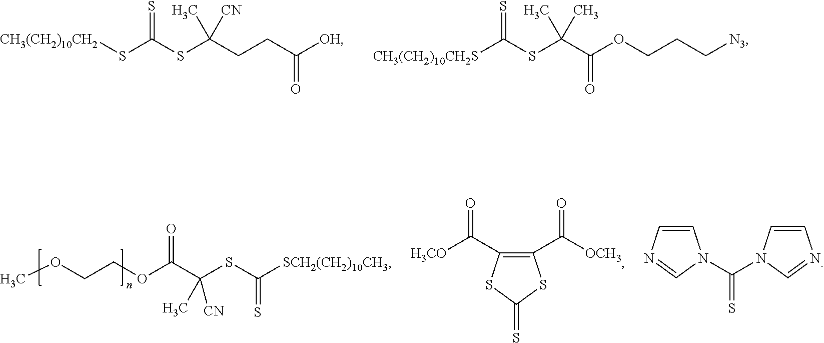

29. The compound of embodiment 11 or 12, wherein one X is NR^{z}, the other X is independently selected from O, S, NR^{z}, and CR³R⁴, and each of R^{z}, R¹, R², R³, and R⁴ is hydrogen.
30. The compound of embodiment 11 or 12, wherein each X is independently NR^{z}, and each of R^{z}, R¹, and R² is independently hydrogen or C₁₋₆ aliphatic.
31. The compound of embodiment 11 or 12, wherein, each X is independently NR^{z}, and each of R^{z}, R¹, and R² is hydrogen.
32. An electrolyte composition comprising a compound of any one of embodiments 11-23 or 29-31.
33. The electrolyte composition of embodiment 32, further comprising a compound of Formula III:

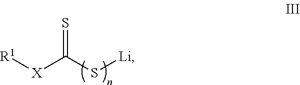

wherein n is 3, 4, 5, 6, 7, or 8.
34. The electrolyte composition of embodiment 32 or 33, further comprising a compound of Formula IV:

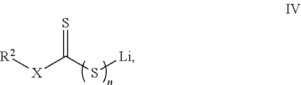

wherein n is 3, 4, 5, 6, 7, or 8.
35. The electrolyte composition of any one of embodiments 32-34, further comprising Li(S)ₙR¹.
36. The electrolyte composition of any one of embodiments 32-35, further comprising Li(S)ₙR².
37. The electrolyte composition of any one of embodiments 32-36, wherein X is S.
38. The electrolyte composition of any one of embodiments 32-37, wherein a lithium trithiocarbonate is the primary (e.g., the greatest percentage by weight or volume) lithium salt in the composition.
39. The electrolyte composition of any one of embodiments 32-38, wherein the electrolyte composition is uncycled.
40. A lithium sulfur battery comprising the binder, additive, or electrolyte of any one of embodiments 1-10 or 24-28 or 32-39.
41. A lithium sulfur battery comprising the compound of any one of embodiments 11-22.
42. A lithium sulfur battery comprising the electrolyte composition of any one of embodiments 32-39.
43. The lithium sulfur battery of any one of embodiments 40-42, wherein the battery is uncycled.
44. A method of making a lithium sulfur battery, comprising the step of adding the binder, additive, or electrolyte of any one of embodiments 1-10 or 24-28, the compound of any one of embodiments 11-23, or the electrolyte composition of any one of embodiments 32-39 to a battery encasement, wherein the step is performed prior to charging or discharging.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. (canceled)

2. An additive for an electrolyte in a lithium sulfur battery comprising a thiocarbonyl functional group.

3. An electrolyte for a lithium sulfur battery comprising a thiocarbonyl functional group.

4. The additive of claim 2, comprising thiocarbonyl functional groups of formula X_{y}C═S, where each X is independently selected from oxygen, nitrogen, sulfur, or carbon, and y is 1 or 2; wherein one X can be taken together with the other X and intervening atoms to form a ring; and when y is 1, X is connected to the thiocarbonyl carbon via a double bond.

5. The additive of claim 4, wherein y is 2.

6. The additive of claim 4, wherein each X is independently selected from nitrogen, sulfur, or carbon.

7. The additive of claim 4, wherein each X is independently selected from sulfur or carbon.

8. The additive of claim 4, wherein y is 1.

9. The additive of claim 8, comprising isothiocyanate functional groups.

10. The electrolyte of claim 3, wherein the electrolyte is uncycled.

11. A compound of Formula I′: 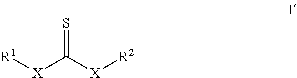
wherein R¹ and R² are each independently hydrogen or an optionally substituted group selected from C₁₋₁₅ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur;
or R¹ and R² are taken together with intervening atoms to form an optionally substituted ring;
each X is absent or independently selected from O, S, NR^{z}, and CR³R⁴;
each R^{z} is independently hydrogen or optionally substituted C₁₋₁₂ aliphatic;
each R³ and R⁴ is independently hydrogen, halogen, —CN, —NO₂, —N(R)₂, —OR, —SR, or an optionally substituted group selected from C₁₋₁₂ aliphatic, 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur; and
each R is independently hydrogen, optionally substituted C₁₋₆ aliphatic, optionally substituted 3- to 7-membered saturated or partially unsaturated carbocyclyl, or optionally substituted 3- to 7-membered saturated or partially unsaturated monocyclic heterocyclyl having 1-3 heteroatoms independently selected from nitrogen, oxygen, and sulfur, or two R when attached to the same nitrogen atom are taken together form an optionally substituted 3- to 7-membered saturated or partially unsaturated monocyclic heterocyclyl having 0-2 additional heteroatoms independently selected from nitrogen, oxygen, and sulfur.

12. (canceled)

13. The compound of claim 11, wherein R¹ and R² are taken together with intervening atoms to form an optionally substituted ring selected from 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

14. The compound of claim 13, wherein R¹ and R² are taken together with intervening atoms to form an optionally substituted 3- to 7-membered saturated or partially unsaturated monocyclic heterocyclyl having 1-3 heteroatoms independently selected from nitrogen, oxygen, and sulfur.

15. The compound of claim 14, wherein R¹ and R² are taken together with intervening atoms to form an optionally substituted 5-membered saturated or partially unsaturated monocyclic heterocyclyl having 2 sulfur heteroatoms.

16. The compound of claim 13, wherein R¹ and R² are taken together with intervening atoms to form an optionally substituted 5- to 6-membered monocyclic heteroaryl having 1-4 heteroatoms independently selected from nitrogen, oxygen, and sulfur.

17. The compound of claim 13, wherein R¹ and R² are taken together with intervening atoms to form Ring A as in Formula II: 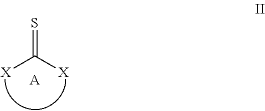
Ring A is an optionally substituted ring selected from 3- to 7-membered saturated or partially unsaturated monocyclic carbocyclyl, 4- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur, phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

18. The compound of claim 17, wherein Ring A is optionally substituted 5-membered saturated or partially unsaturated monocyclic heterocyclyl having 2 sulfur heteroatoms.

19. The compound of claim 17, wherein Ring A is optionally substituted 5- to 6-membered monocyclic heteroaryl having 1-4 heteroatoms independently selected from nitrogen, oxygen, and sulfur.

20. The compound of claim 11, wherein both X are absent.

21. The compound of claim 11, wherein both X are S.

22. The compound of claim 11, wherein both X are NR².

23. The compound of claim 11, wherein the compound is trithiocyanuric acid.

24-28. (canceled)

29. The compound of claim 11, wherein one X is NR^{z}, the other X is independently selected from O, S, NR^{z}, and CR³R⁴, and each of R^{z}, R¹, R², R³, and R⁴ is hydrogen.

30. The compound of claim 11, wherein each X is independently NR^{z}, and each of R^{z}, R¹, and R² is independently hydrogen or C₁₋₆ aliphatic.

31. The compound of claim 11, wherein, each X is independently NR^{z}, and each of R^{z}, R¹, and R² is hydrogen.

32-44. (canceled)

45. The additive of claim 4, wherein each X is nitrogen.

46. The electrolyte of claim 3, comprising thiocarbonyl functional groups of formula X_{y}C═S, where each X is independently selected from oxygen, nitrogen, sulfur, or carbon, and y is 1 or 2; wherein one X can be taken together with the other X and intervening atoms to form a ring; and when y is 1, X is connected to the thiocarbonyl carbon via a double bond.

47. The electrolyte of claim 46, wherein each X is independently selected from nitrogen, sulfur, or carbon.

48. The electrolyte of claim 46, wherein each X is nitrogen.
